(19) United States
(12) Patent Application Publication
(10) Pub. No.: US 2026/0021326 A1
(43) Pub. Date: Jan. 22, 2026

(54) COMPACT ARC THERAPY SYSTEMS AND METHODS

(71) Applicant: The New York Proton Center, New York, NY (US)

(72) Inventors: Minglei KANG, New York, NY (US); Xingyi ZHAO, New York, NY (US); J. Isabelle CHOI, New York, NY (US); Haibo LIN, New York, NY (US); Charles B. SIMONE, New York, NY (US)

(73) Assignee: The New York Proton Center, New York, NY (US)

(21) Appl. No.: 19/273,845

(22) Filed: Jul. 18, 2025

Related U.S. Application Data
(60) Provisional application No. 63/674,096, filed on Jul. 22, 2024.

(51) Int. Cl. A61N 5/10

(52) U.S. Cl. CPC A61N 5/1067; A61N 5/1081; A61N 2005/1087

(57) ABSTRACT

Radiotherapy is the most widely used and effective anti-tumor therapy, however it can damage healthy tissues surrounding the tumor. One approach to reducing damage to healthy tissue involves irradiation at dose rates far exceeding those currently used in clinical contexts reduces radiation-induced toxicities while maintaining an equivalent tumor response. This is known as the FLASH effect. The mechanism responsible for reduced tissue toxicity following FLASH radiotherapy (FLASH-RT) is still undetermined; multiple hypotheses have been suggested by linking the high dose rate to rapid oxygen depletion, immune response, reduction of peroxyl radical lifetime, preservation of normal tissue stem cells, etc. While effective, proton therapy is underutilized, contributing to fewer than 2% of all external beam radiation treatments. To overcome this shortcoming, provided are methods which utilize the highest energy layer and a universal range shifter (URS) to facilitate rapid spot arc therapy (RAPIDSPARC™).

### INCORPORATION BY REFERENCE OF RELATED APPLICATION

This application claims priority of U.S. Provisional Patent Application No. 63/674,096 filed Jul. 22, 2024, which is incorporated by reference herein in its entirety.

### TECHNICAL FIELD

The present disclosure generally relates to systems and methods for providing radiotherapy.

### BACKGROUND

Radiotherapy (RT) is required by 60-70% of cancer patients during their treatment. Radiotherapy is the most widely used and effective anti-tumor therapy, however it can damage healthy tissues surrounding the tumor. Thus, preventing or mitigating radiation-induced healthy tissue injury has always been a topic of particular interest in radiotherapy research.

One approach to reducing damage to healthy tissue involves irradiation at dose rates far exceeding those currently used in clinical contexts reduces radiation-induced toxicities while maintaining an equivalent tumor response. This is known as the FLASH effect. The mechanism responsible for reduced tissue toxicity following FLASH radiotherapy (FLASH-RT) is still undetermined; multiple hypotheses have been suggested by linking the high dose rate to rapid oxygen depletion, immune response, reduction of peroxyl radical lifetime, preservation of normal tissue stem cells, etc.

Recently, there has been growing interest in using proton therapy to conduct FLASH-RT translational research. Transmission/shoot-through beams have been proposed for FLASH proton treatment planning.

While effective, proton therapy is underutilized, contributing to fewer than 2% of all external beam radiation treatments. This is largely due to the high costs involved in establishing and operating proton therapy centers, including the expenses for building, equipping, and maintaining these facilities. Such financial barriers limit availability, particularly in regions with less healthcare funding.

The technologies used to deliver photon-based treatments have progressed a great deal over the last few decades, evolving from 3D conformal radiotherapy to intensity-modulated radiotherapy (IMRT) to volumetric modulated arc therapy (VMAT), in which the linear accelerator delivering the therapeutic X-rays rotates around the patient during treatment. Each of these steps helped to improve dose coverage of the tumor being targeted, while reducing unwanted radiation being delivered to normal tissues within the body. First described in 1997, in proton arc therapy (PAT) protons are delivered continuously as a gantry—the large circular structure containing the equipment that delivers the protons to the patient—rotates around the patient. This improves dose distribution and increase treatment robustness compared with IMPT.

Still, the current approach to proton arc therapy involves using either multiple energy layers per sub-arc or a mono-energy per sub-arc (ARC), depending on the energy switching capabilities of cyclotron or synchrotron accelerators. This method often restricts the efficiency of treatment delivery. Disclosed are methods which utilize the highest energy layer and universal range shifter (URS) to facilitate rapid spot arc therapy (RAPIDSPARC™).

### SUMMARY

In a first embodiment, provided is a system for administering at least two fields of shifted and compensated ionizing radiation to a target tissue, the system including:
a. an ionizing radiation source configured to produce single-energy beams to form at least two fields of shifted and compensated ionizing radiation;
b. a treatment head configured to deliver the single-energy beams to the target tissue, wherein the treatment head includes:
   i. at least one universal range shifter adjusted to shift a range of the single-energy beams so that the Bragg peak of the single-energy beams coincides with the target tissue; and
   ii. at least one range compensator adjusted to compensate the range of the single-energy beams so that the Bragg peak of the single-energy beams coincide with a contour of the target tissue.

In embodiments, the system does not include an energy selection system.

In embodiments the range shifter includes multiple plates that reduce the range of the ionizing radiation, and combinations of the range shifters are calculated by applying parameters determined using an inverse-planning optimization algorithm, wherein said parameters include the number and location of the plates through which the ionizing radiation is transmitted.

In some embodiments the range compensator contours are calculated by applying parameters determined using an inverse-planning optimization algorithm. In embodiments, the at least two fields of the shifted and compensated ionizing radiation comprises three fields, or four fields, or five fields, or more.

Further embodiments provide methods for administering at least two fields of shifted and compensated ionizing radiation to a target tissue, said methods including use of disclosed systems, the systems including an ionizing radiation source configured to produce single-energy beams to form at least two fields of shifted and compensated ionizing radiation; a treatment head configured to deliver the single-energy beams to the target tissue, wherein the treatment nozzle includes at least one universal range shifter adjusted to shift a range of the single-energy beams so that Bragg peak of the single-energy beams coincides with the target tissue, and at least one range compensator adjusted to compensate the range of the single-energy beams so that the Bragg peak of the single-energy beams coincide with a contour of the target tissue.

In embodiments, the method does not include use of an energy selection system.

Further embodiments include a method for supplying a field of ionizing radiation to a target tissue, which includes providing an ionizing radiation, forming at least two fields of shifted and compensated ionizing radiation by shifting the range of the ionizing radiation by passing the ionizing radiation through an adjustable range shifter, so that the Bragg peak of the ionizing radiation coincides with the target tissue; and compensating the range of the ionizing radiation by passing the ionizing radiation through an adjustable range compensator, so that the Bragg peak of the ionizing radiation coincides with the target tissue; and directing to the target tissue at least two fields of the shifted and compensated ionizing radiation to provide a uniform dose distribution across a target volume.

Embodiments may include administration of a desired dose rate such as a dose rate of at least 10 Gy/s, at least 20 Gy/s, at least 40 Gy/s, at least 60 Gy/s, or more. The first embodiment may include administration of shifted and compensated ionizing radiation that does not substantially extend proximally beyond a distal edge of the target location. The embodiment may include compensated ionizing radiation composed of protons, helium, carbon, argon or neon. The embodiment may include compensated ionizing radiation composed of protons.

In some embodiments the target location is cancerous tissue. In some embodiments the range shifter includes multiple plates that reduce the range of the ionizing radiation, and said combinations of the range shifters are calculated by applying parameters determined using an inverse-planning optimization protocol, wherein said parameters comprise the number and location of the plates through which the ionizing radiation is transmitted. In embodiments the range compensator contours are calculated by applying parameters determined using an inverse-planning optimization protocol. In some embodiments, inverse-planning optimization determines the distribution parameters of the ionizing radiation, and/or determines the weighting parameters of the ionizing radiation. In some embodiments, the shifted and compensated ionizing radiation includes three, four, or five fields of the shifted and compensated ionizing radiation.

A further embodiment comprises a system for producing at least two shifted, compensated fields of particle beams at a desired dose rate, such as a dose rate of at least 10 Gy/s, at least 20 Gy/s, at least 40 Gy/s, at least 60 Gy/s, at least 40 Gy/s, at least 40 Gy/s, said system including an ionizing radiation source configured to produce a particle beam; a universal range shifter configured to adjustably shift the range of the particle beam; and a range compensator configured to adjustably compensate the range of the particle beam. In embodiments the particle beams include protons, helium, carbon, argon, or neon.

In embodiments, the system does not include an energy selection system.

Another embodiment comprises a method of treating a target tissue, including diagnosing a target tissue; mapping the target tissue; developing a radiotherapy treatment plan to administer an effective amount of shifted and compensated ionizing radiation to the target tissue; and shifting and compensating an ionizing radiation using a system including an ionizing radiation source configured to produce a particle beam; a universal range shifter adjusted to shift the range of the proton/particle beam so that the Bragg peak of the particle beam coincides with the target tissue; a range compensator adjusted to compensate the range of the particle beam so that the Bragg peak of the particle beam coincides with the contour of the target tissue; and then administering the particle beam to the target tissue. In embodiments the particle beam is applied in a dose rate of at least 40 Gy/s. In embodiments the target tissue includes a neoplasm or benign tumor. In embodiments the particle beam does not substantially extend proximally beyond a distal edge of the neoplasm or benign tumor. In embodiments wherein the range shifter includes multiple plates that reduce the range of the ionizing radiation, and the range shifter plate positioning is determined by applying parameters determined using an inverse-planning optimization protocol, wherein said parameters comprise the number and location of the plates through which the ionizing radiation is transmitted. In embodiments, the shape of said range compensator is calculated by applying parameters determined using an inverse-planning optimization protocol. In embodiments, the at least two fields of the shifted and compensated ionizing radiation comprises three fields, or four fields, or five fields. In embodiments the particle beams include protons, helium, carbon, argon, or neon.

In embodiments, the method does not include use of an energy selection system.Another embodiment comprises a method of adjusting a proton therapy device including receiving a treatment plan designed to apply FLASH-RT to a target location, wherein said treatment plan comprises a target location, a three-dimensional target shape, a number of treatment fields, and target dose rate of at least 40 Gy/s; and modifying the energy or range of the proton therapy device using a range shifter and a range compensator, wherein said range shifter comprises multiple plates that reduce the range of the ionizing radiation, wherein said modifying comprises use of an inverse-planning protocol to determine range shifter and range compensator parameters so that the Bragg peak of the energy output of the proton therapy device coincides with a target tissue, and wherein said parameters comprise the number and location of the plates through which the energy or range of the proton therapy device is transmitted. In embodiments the energy output of the proton therapy device does not substantially extend proximally beyond a distal edge of the cancerous tissue.

Another embodiment includes a method for treating a cancerous tissue comprising; providing an ionizing radiation transmission beam with a dose rate of at least 40Gy/s; adjusting the energy or range of the ionizing radiation transmission beam such that the Bragg peak of the beam coincides with a point between 3 mm and 5 mm from an edge of the cancerous tissue; and applying the ionizing radiation transmission beam to the cancerous tissue. In an embodiment the ionizing radiation transmission beam includes protons, helium, carbon, argon, or neon.

In another embodiment, provided is a system for administering PAT comprising at least two fields of shifted and compensated ionizing radiation to a target tissue, the system including:
a. an ionizing radiation source configured to produce single-energy beams to form at least two fields of shifted and compensated ionizing radiation;
b. a treatment head configured to deliver the single-energy beams to the target tissue, wherein the treatment head includes:
   i. at least one universal range shifter adjusted to shift a range of the single-energy beams so that the Bragg peak of the single-energy beams coincides with the target tissue.

Further embodiments comprise methods of treating cancerous tissue comprising PAT comprising administration of proton radiation sequentially from multiple angles.

Disclosed PAT embodiments utilize a single-energy layer, providing ultra-fast delivery and eliminating the extended energy switching time (often exceeding one second). In embodiments, a URS composed of variable thickness plates effectively adjusts the range of the single-energy beam for each sub-arc, facilitating rapid delivery. By incorporating a Genetic Algorithm (GA) to optimize the sequence of range layers for each sub-arc, along with greedy spot map optimization and Linear Energy Transfer (LET) optimization, disclosed embodiments achieve plan quality comparable to multiple-energy layer approaches while enhancing LET for critical Organs at Risk (OARs). This novel approach has been evaluated using a C-shaped phantom target and clinical case studies. The plan quality has been benchmarked against standard Intensity-Modulated Proton Radiotherapy (IMPT) and ARC, demonstrating its efficacy.

### BRIEF DESCRIPTION OF THE FIGURES

FIG. 1 shows a schematic diagram of non-transmission FLASH intensity-modulated particle therapy (IMPT) planning using universal range shifter (URS) and range compensators (RC). The URS and RC are placed in the beam path for illustration purpose only; they can be placed in various order depending on treatment goals.

FIG. 2 shows that an example of spot distribution and weight optimization can effectively improve the plan quality and FLASH-RT dose rate distribution.

FIG. 3 shows transmission (FIG. 3(a) & FIG. 3(d)) vs. Bragg peak (FIG. 3(b) & FIG. 3(e)) planning using 250 MeV proton beams for C-shape target in a water phantom.

FIG. 4 shows the dose comparisons between transmission and Bragg peak plans for three selected lung patients using the same beam arrangement. The right and middle columns represent transmission and Bragg peak plans, respectively.

FIG. 5 shows dose rate comparisons between transmission (the left side images) and Bragg peak (the middle images) plans using the same beam arrangement.

FIG. 6 upper view: the single spot (1000MU/spot) 2-D dose rate distribution for 250 MeV proton beam at central axis plane evolves in water phantom with air gaps of 5, 15, and 25 cm, respectively; lower view: the spot dose rate at the central axis (the three sections represent the 20 cm transport in water, air gaps, and the residual range in water).

FIG. 7 shows an example illustrating beam angle optimization. FIG. 7(a) and FIG. 7(b) are the 2D dose distribution using different fields and field angles, FIG. 7(c) and FIG. 7(d) are the DVH, and DRVH comparison, FIG. 7(e) is the V40Gy/s dose rate coverage vs. OAR doses from the low to high dose regions. The left side lung and most of the heart are completely spared using a beam arrangement shown by FIG. 7(b).

FIG. 8 shows an exemplary Range Compensator (RC).

FIG. 9 shows the dosimetric comparison between Bragg peak and conventional IMPT plans for 10 liver cancer patients. FIG. 9(a) Liver-GTV Dₘₑₐₙ, FIG. 9(b) heart D_{0.5cc} , FIG. 9(c) chest wall D_{2cc}, and FIG. 9(d) CTV Dₘₐₓ for SBRT, Bragg-400MU, and Bragg-800MU. n.s. represents that the results are statistically non-significant (p≥0.05). The interquartile (25-75th percentile) is denoted by the ends of the box, the median is represented by a horizontal line inside of the box, and the highest and lowest values are denoted by two lines outside of the box. The diamond marker indicates data points beyond the 25-75^{th} percentile. Liver treatment planning studies demonstrated that the novel single-energy PBS delivery method can achieve a similar or equivalent plan quality compared to the conventional multi-energy proton PBS plans.

FIG. 10 shows a comparison of current design proton therapy systems (“current design”) and a system as disclosed herein (“novel design”) illustrating the structural differences between FIG. 10(a) a conventional design and FIGS. 10(b,c) a disclosed compact system for delivering both FLASH and conventional dose rate RT using the highest energy of the cyclotron. The difference between “upstream” (current practice using an energy selection system) and “downstream” (disclosed herein) energy modulation as well as transmission planning is also shown.

FIG. 11 shows FIG. 11(a) pseudo-CT image and FIG. 11(b) 3D model of a disclosed range compensator.

FIG. 12 shows an end-to-end demonstration of single-energy proton Bragg peak RT as disclosed herein, encompassing all stages from treatment planning and delivery to precise dose and dose rate measurements.

FIG. 13 shows representative treatment plans for FIG. 13(a) a solid water phantom and FIG. 13(b) a head and neck patient phantom.

FIG. 14 shows dose and dose rate calculation comparisons between PCS and MC using a homogeneous phantom. FIGS. 14(a-b) the 2D dose distribution (coronal-section view) for one of the three beams calculated by PCS and MC, FIGS. 14(c-d) PCS and MC 2D dose distribution with the spot map, FIG. 14(e) line dose profiles of the PCS and MC, FIGS. 14(f-g) PCS and MC 2D dose rate distribution with the spot map, FIG. 14(h) line profiles of the PCS and MC dose rate.

FIG. 15 shows a comparison of dose and dose rate calculation between PCS and MC using a head-neck phantom. FIGS. 15(a-b) coronal view of the head-neck lateral beam dose distribution using PCS and MC, FIGS. 15(c-d) PCS and MC dose distribution with the reduced spot map, FIG. 15(e) line dose profiles comparison of the PCS and MC, FIGS. 15(f-g) dose rate distribution with the spot map, FIG. 15(h) line dose profiles comparison of the PCS and MC.

FIG. 16 shows a dose comparison between PCS and measurement using a solid water phantom at 3 depths, the left column is the TPS dose distribution, the left second column is the measured dose distribution, the third column from the left is the gamma comparison, and the right column is the line profile comparisons. FIGS. 16(a-d), FIGS. 16(e-h) and FIGS. 16(i-l) are the results for depths of 15,12 and 9 cm, respectively.

FIG. 17 shows a dose rate measured distribution using a hybrid method under beam currents 100 and 215 nA of the field G90C0 for solid water (SW) (a, b) and head neck (HN) phantom (c, d).

FIG. 18 shows a dose recontruction of the FIG. 17(a) plan and FIG. 17(b) measurement for field G9000 of the head-neck case. FIG. 17(c) The gamma passing rate distribution and FIG. 17(d) line profile comparison between the plan and measurement.

FIG. 19 shows the pristine Bragg peak differences between the URS pulled back (a) and the cyclotron-based mono energy proton (b).

FIG. 20 shows an illustrative diagram of the quasi-distal tracking method used in arc therapy. FIG. 20(a) depicts the configuration of sub-arcs that collectively form the arc therapy treatment. FIG. 20(b) provides a detailed view of the geometric relationship between various elements: the range of each sub-arc, the quasi-distal tracking margins (denoted as r_proximal and r_distal), and the width of the lateral geometry of the sub-arc, labeled as w_n.

FIG. 21 shows the dosimetry comparison among conventional IMPT FIG. 21(a), ARC FIG. 21(b), and the novel RAPIDSPARC™ FIG. 21(c). The DVH in FIG. 21(d) uses dot lines to represent ARC, dashed lines for RAPIDSPARC™, and solid lines for IMPT. While target coverage is consistent across all three techniques, RAPIDSPARC™ exhibits a comparable core dose, but a reduced dose compared to IMPT. The LET histogram in FIG. 21(d) shows that RAPIDSPARC™ has the lowest LET compared to both IMPT and ARC.

FIG. 22 shows dosimetry and Dose-averaged LET volume histograms (LVHs) comparison for a selected prostate patient using ARC and RAPIDSPARC™. FIG. 22(a) and (b) 2D dose distribution of ARC and RAPIDSAPRC™, (c) shows the dose-volume histogram (DVH) comparison, (d) and (e) are the LET distribution, (f) is the LVHs comparison.

FIG. 23 shows 2D dose distributions for a selected head and neck patient using SIB to deliver 59.5 and 70 Gy for conventional IMPT FIG. 23(a), ARC FIG. 23(b), and RAPIDSPARC™ FIG. 23(c). Panel FIG. 23(d) showcases the DVH comparison for all three techniques.

FIG. 24 shows a dose rate distribution among ARC and RAPIDSPARC™ techniques. FIG. 24(a) and FIG. 24(b) are the 2D dose rate distribution, FIG. 24(c) is the line dose rate profile comparison.

TABLE 1 shows dosimetry and dose rate coverage of V40Gy/s comparison for transmission and Bragg peak IMPT plans for all six lung cases. The dosimetry comparison used RTOG 0915 metrics. Both dose and dose rate statistics used the averaged values for all six cases. The last row of the table represents the averaged V40Gy/s for both target and OARs.

TABLE 2 shows how an exemplary URS's six range shifter plates can generate 34different combinations to “pull back” or reduce the proton range between 0 cm to 34 cm with a step of 1 cm. Here, “1” represents that the plate will be moved into the beam path to pull back the proton beam range, and “0” means that plate won't be used to pull the range back.

TABLE 3 shows plan quality comparisons between Bragg Peak and conventional IMPT plans for 10 lung cancer patients. Lung treatment planning studies demonstrated that the novel single-energy PBS delivery method can achieve a similar or equivalent plan quality compared to the conventional multi-energy proton PBS plans. p1, p-values of the two-tailed student t-tests between BP-1200MU-2 ms vs IMPT-SBRT plans; p2, p-values of the two-tailed student t-tests between BP-300MU-0.5 ms vs IMPT-SBRT plans.

TABLE 4 shows dosimetric characteristics for 3 techniques adhering to clinical constraints in 6 head and neck cancer patient.

### DETAILED DESCRIPTION

FLASH-RT improves patient outcomes but requires specialized hardware and expertise. Further, current transmission/shoot-through plans do not utilize Bragg peaks for dose delivery, which results in unnecessary irradiation exposure to normal tissues distal to the target volume. In the present disclosure “distal” to refers to further along a beam path, as opposed to “anatomically” distal.

Single-energy pencil beam scanning (PBS) delivery methods disclosed herein can achieve a similar or equivalent plan quality compared to conventional multi-energy proton PBS plans. By eliminating the expensive energy selection systems and beam focusing systems, the proton treatment cost will be significantly reduced and make PBS FLASH-RT more affordable for the public. Use of a single energy-layer proton beam from a cyclotron for conformal conventional dose rate/FLASH-RT can be a promising solution for future proton system design.

Disclosed embodiments comprise commercially-available equipment modified with additional components to utilize the Bragg peak-a pronounced peak on the Bragg curve which plots the energy loss of ionizing radiation during its travel through matter. For protons, a-rays, and other ion rays, the peak occurs immediately before the particles come to rest. It will be appreciated that the Bragg curve for these sorts of particles are qualitatively different than those for x-rays or other types of electromagnetic radiation.

Disclosed methods and systems include FLASH-RT tumor treatment modalities that modify currently-available proton and other charged heavy particles (for example helium, carbon, argon and neon) systems utilizing methods based on an inverse optimization algorithm that requires minimal hardware modification to utilize the proton beam Bragg peak region to treat tumors. The Bragg peak is a pronounced peak on the Bragg curve which plots the energy loss of ionizing radiation during its travel through matter.

The Bragg peak can be identified using a graphic representation of the energy of certain charged particles such as protons-energy lost by certain charged particles is inversely proportional to the square of their velocity, thus the graphed peak occurs just before the particle comes to a complete stop. By correlating the Bragg peak with the target tissue, disclosed methods and systems avoid the “exit dose” transmitted far beyond a target tissue and, therefore, spare tissue adjacent to target areas. The Bragg peak can be calculated using commercially available software.

Disclosed systems include adjustable, universal range shifter (URS) comprising materials such as plastic plates that “pull back” or reduce the energy of ionizing radiation beams, and thus reduce the range of the beams. By providing a number of “plates” of various thicknesses at discrete distances in the beam path, disclosed systems allow a user to accurately and reproducibly reduce the range of the beam to a target depth in tissue using ionizing radiation sources that would otherwise be unsuitable for FLASH-RT utilizing the Bragg peak of the particles comprising the ionizing radiation.

Disclosed systems include range compensators (RC) comprising materials that further modify the range of ionizing radiation beams, and thus tailor the penetration depths of the beams to deliver conformable radiation to target tissue. For example, disclosed RCs comprise a three-dimensinal contour to finely adjust the range of the range-shifted ionizing radiation (an exemplary RC is shown in FIG. 8). By providing range compensators of various thicknesses in beam direction and covering the target tissue laterally using scanned discrete proton spots for the determination of the optimal 3-D shape, disclosed systems allow a user to accurately and reproducibly conform the beam to the distal target shape using ionizing radiation sources that would other wise be unsuitable for FLASH-RT using the Bragg peak of the particles comprising the ionizing radiation. The RC can finely adjust the ionizing radiation before, during, or after the radiation is range-shifted.

Disclosed methods comprise adjusting the range of an energy beam, for example a proton beam, and compensating proton ranges to target an area of target tissue such that the Bragg peak of the beam coincides with the target tissue, for example the distal area of target tissue, or in some cases immediately beyond the distal edge of the target tissue, thus reducing or eliminating the “exit” dose of proton beams that extend beyond the target, while still preserving FLASH-RT effectiveness. As a result of the rapid drop-off in particle energy at the penetration range reflected by the Bragg peak, the majority of the particle energy can be limited to the target tissue.

In intensity-modulated radiation therapy (IMRT) such as intensity-modulated particle therapy (IMPT), the beam intensity is varied across each treatment region (target) in a patient. Depending on the treatment modality, parameters available for intensity modulation include beam shaping (collimation), beam weighting (spot scanning), and angle of incidence (beam geometry). These degrees of freedom lead to an effectively infinite number of potential treatment plans. Therefore, consistently and efficiently generating and evaluating high-quality treatment plans relies on the use of computing systems.

In embodiments, an inverse planning tool was developed to optimize IMPT using a single-energy layer for FLASH-RT planning or non-FLASH dose rate planning. Inverse planning is the process by which the intensity distribution of each beam employed in a treatment plan is determined such that the resultant dose distribution can best meet the criteria specified by the planner. In inverse planning, a radiation oncologist defines a patient's critical organs and tumor volume, after which they also determine target doses and importance factors for each. Then, a planner runs an optimization program to find the treatment plan that best matches all the input criteria. Thus, inverse planning uses the optimizer to solve the Inverse problem as set up by the planner. The range pulling-back vaues and physical compensator contours may be calculated to stop single-energy proton beams at the distal edge of the target.

In embodiments the spot map and weights of each field were optimized to achieve a sufficient dose rate using proton beam Bragg peaks. Treatments of both “phantom” (model) and patients are planned using disclosed methods and transmission techniques to assess dosimetry and dose rate characteristics.

Disclosed methods comprise use of single-energy Bragg peak delivery. Disclosed embodiments utilize the highest energy protons (or helium, carbon, argon, and neon) directly from the accelerator, eliminating the need for “upstream” (for example, between the radiation source and the bending magnets) energy selection systems. Disclosed Bragg peak FLASH approaches, using “pristine” beams in PBS mode with inverse plan optimization, offer high conformity in treatment. Studies in various treatment sites, including the lung, liver, partial breast, head-neck, and prostate areas, have shown superior dosimetry characteristics compared to conventional multi-energy IMPT, achieving dose rates above 40 Gy/s in OARs.

In some systems, the energy of the proton beams produced by the cyclotron were reduced and the “spectrum” of the proton beams was purified or filtered by an energy selection system prior to reaching the patient treatment room. This increased treatment times as the proton source required adjustment between beam “shots.” In contrast, disclosed systems employ a cost-effective, compact design with beam-specific range pullbacks utilizing URS and RC in the treatment head, eliminating the “upstream” energy conditioning of previous systems, drastically reducing treatment times and reducing system size and cost. In embodiments, the URS range reduction increments can be, for example, 0.1 cm, 0,15 cm, 0.25 cm, 0.5 cm, 0.75 cm, 1.0 cm, or the like.

In disclosed PAT embodiments, methods of treating cancerous tissue comprise proton arc therapy comprising administration of proton radiation sequentially from multiple angles.

Disclosed PAT embodiments utilize a single-energy layer, which features the highest beam current, providing ultra-fast delivery and eliminating the extended energy switching time (often exceeding one second). In embodiments, a URS composed of variable thickness plates effectively adjusts the range of the single-energy beam for each sub-arc, facilitating rapid delivery.

### Definitions

“Administration,” or “to administer” means the step of giving (i.e. administering) a treatment to a subject.

“Bragg Curve” means a graph of the energy loss rate of a particle, or Linear Energy Transfer (LET), as a function of the distance through a stopping medium. The energy loss is characterized primarily by the square of the nuclear charge, Z, and the inverse square of the projectile velocity, B. This gives the Bragg Curve its shape, “peaking” (and thus showing the range at which a particle is releasing most of its energy) just before the projectile stops.

“Bragg Peak” is a pronounced peak on the Bragg Curve which plots the energy loss of ionizing radiation during its travel through matter. The Bragg peak identifies the range at which a particle releases most of its energy.

“Calculate” refers to the selection and adjustment of the range shifters and/or range compensator to determine the positioning and combinations of the plates to produce an ionizing radiation of the desired range.

“Cancerous tissue” means any neoplasm or benign tumor.

“Energy selection system” refers to the energy degradation systems/devices that reduce the energy of the beam from the cyclotron as well as the purification system/devices that purify the spectrum of the beam. Typically the energy selection system is placed outside the treatment room. “Energy selection system” does not include URS or RC.

“Field” means an area treated by an ionizing radiation beam at a particular angle. Radiotherapy treatment can be delivered using a single field or multiple fields at different angles.

“FLASH radiotherapy” or “FLASH-RT” is a radiotherapy treatment method that delivers radiation dose with ultra-high dose rates.

“Particle beams” refers to an ionizing radiation comprising protons or other charged particles, for example helium, carbon, argon and neon.

“Patient” means a human or non-human subject receiving medical or veterinary care.

“Plate” as used herein refers to a URS or RC component (for example, polycarbonate plastic) through which an ionizing radiation is passed to lower the energy of the ionizing radiation or to reduce the penetration ranges, or to finely adjust the beam pentetration ranges of the ionizing radiation.

“Proton arc therapy” or “PAT” means administration of multiple proton radiation beams from multiple angles.

“Range Compensator” or “RC” means hardware configured to finely adjust or reduce the the range of an ionizing radiation into a beam form suitable for administration for Bragg peak-based FLASH-RT as described herein. Range compensators finely adjust the range of the shifted ionizing radiation to account for the three-dimenional shape of the target tissue.

“Single energy beam” means that the energy of the beam from the ionizing radiation source (such as a cyclotron) is not adjusted during a treatment session; successive beam “shots” employ the same energy with range and contour adjustment occurring via URS and RC in the treatment head.

“Target tissue” refers to the tissue to be treated, for example cancerous tissue such as a cancerous tissue including neoplasms and benign tumors.

“Tuned” as used herein means to optimize the number of spots, weightings of spots, and locations of spots during inverse optimization to determine spot map to reach an optimal plan.

“Universal Range Shifter” or “URS” means hardware configured to adjust/reduce the range of an ionizing radiation range into a form suitable for administration for Bragg peak-based FLASH-RT as described herein.

### Administration Systems

Some embodiments disclosed herein comprise administration systems, such as administration systems for administering RT treatments. In embodiments, disclosed systems comprise a source of ionizing radiation, for example, a cyclotron or a synchrotron. In embodiments, the ionizing radiation source emits an ionizing radiation, for example protons, helium, carbon, argon and neon, or other heavy particles.

Disclosed embodiments comprise systems for administering at least two fields of shifted and compensated ionizing radiation to a target tissue, the system including:
a. an ionizing radiation source configured to produce single-energy beams to form at least two fields of shifted and compensated ionizing radiation;
b. a treatment head configured to deliver the single-energy beams to the target tissue, wherein the treatment head includes:
   i. at least one universal range shifter adjusted to shift a range of the single-energy beams so that the Bragg peak of the single-energy beams coincides with the target tissue; and
   ii. at least one range compensator adjusted to compensate the range of the single-energy beams so that the Bragg peak of the single-energy beams coincide with a contour of the target tissue.

In embodiments, the administration system does not include an energy selection system.

Some disclosed embodiments further comprise range shifters. For example, in disclosed embodiments, range shifters can comprise a universal range shifter (URS), that can convert an ionizing radiation into fields of discrete range. In disclosed embodiments, the URS can comprise multiple plastic plates, for example transparent amorphous thermoplastic plates such as polycarbonate plastic plates, with varying thicknesses. In embodiments, the ranges of the separate fields can be shifted by differing amounts. For example, in an embodiment employing 5 fields for treatment, the five fields may comprise 1, 2, 3, 4, or 5 different range shifts, thus 1, 2, 3, 4, or 5 different URS plate combinations.

Some disclosed system embodiments further comprise range compensators (RC). For example, in disclosed embodiments, the RC can comprise at least one plastic plate with contours having various thicknesses, for example a solid, transparent amorphous thermoplastic plate such as a polycarbonate plastic plate. In embodiments, the plates can be of a density of, for example, 0.5 g/cm³, 0.6 g/cm³, 0.7 g/cm³, 0.8 g/cm³, 0.9 g/cm³, 1.0 g/cm³, 1.1 g/cm³, 1.2 g/cm³, 1.3 g/cm³, 1.4 g/cm³, 1.5 g/cm³, 1.6 g/cm³, or the like. In embodiments, the RC can further refine the range of the ionizing radiation that has been range-shifted with the URS.

In embodiments, the range of the separate fields can be altered by different RCs. For example, in an embodiment employing 5 fields for treatment, the five fields may comprise 1, 2, 3, 4, or 5 different RCs. An exemplary RC is shown in FIG. 8, with the 3-D “topography” of the RC clearly visible.

When combined with disclosed URS, RC, and inverse planning embodiments, this allows for the provision of Bragg peak-utilizing FLASH-RT treatments using conventional proton RT equipment, for example by modifying the beam range.

In embodiments, the administration system does not comprise an energy selection system. Rather, the specific range and contour of the beam is determined by the range shifter(s) and the range compensator(s). In embodiments, the URS and RC are located in the treatment head or nozzle.

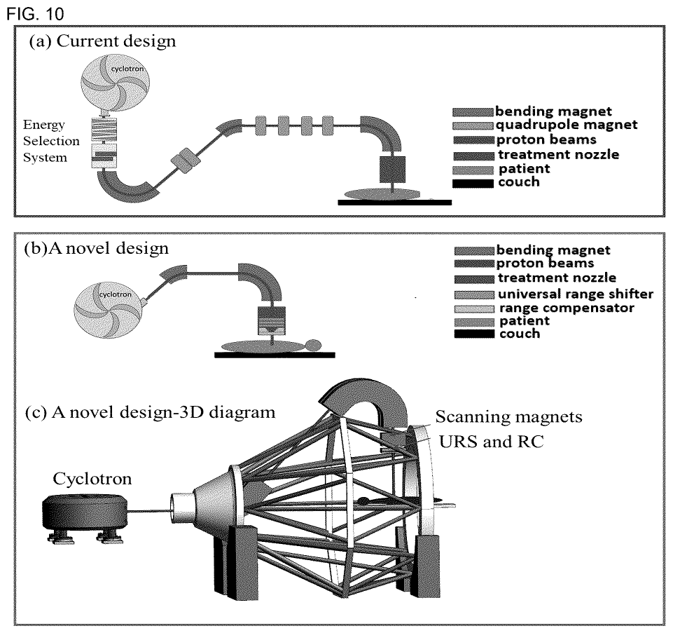

For example, in the system of FIG. 10(a) employed an energy selection system located just “downstream” of the cyclotron, as well as URS and RC in the treatment head or nozzle. In contrast, as seen in FIG. 10(b), disclosed systems employ only URS and RC to modulate the range and contour of the energy beam.

Further disclosed embodiments comprise systems for administering PAT comprising at least two fields of shifted ionizing radiation to a target tissue, the system including:
a. an ionizing radiation source configured to produce single-energy beams to form at least two fields of shifted and compensated ionizing radiation;
b. a treatment head configured to deliver the single-energy beams to the target tissue, wherein the treatment head includes:
   i. at least one universal range shifter adjusted to shift a range of the single-energy beams so that the Bragg peak of the single-energy beams coincides with the target tissue.

In embodiments, the PAT administration system does not comprise a range compensator.

In embodiments, the PAT administration system URS adjusts the proton range for each beam angle.

In embodiments, the PAT administration system does not comprise an energy selection system. By eliminating the need for an energy selection system, disclosed embodiments reduce the cost and bulk of RT systems. Further, because the ionizing radiation source is providing a single energy beam without adjustment between successive beam “shots”, the time required for effective treatment is greatly reduced.

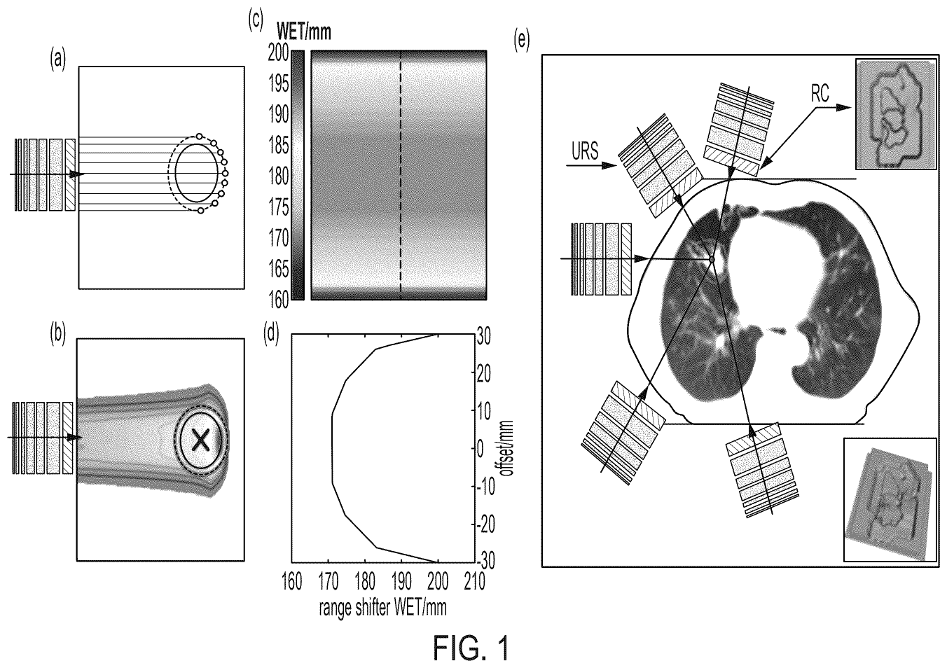

Some disclosed embodiments comprise inverse-planning to target the ionizing radiation. For example, in embodiments, ray tracing is used to calculate the range compensation. In embodiments, an energy beam is customized to generate the intensity-modulated spot map via the inverse planning platform. For example, as shown in FIG. 1(a), a uniform margin of, for example, 6-mm on the CTV can be used to contain the spot distribution in-depth direction. The 90% of dose falloff can be used as the proton range for spot map generation. The water equivalent thickness (WET) of each pencil beam proton radiographic track (denoted by WETi (x, y, z)) can be calculated by Eq.1, and rsp(x, y, z) represent the relative stopping power (rsp) of each voxel of the 3D CT images.

The integral step in Eq.1 can be accurately computed with a raytracing algorithm (Siddon RL. Prism representation: A 3D ray-tracing algorithm for radiotherapy applications. Phys. Med. Biol. 1985; 10.1088/0031-9155/30/8/005 30 (8), 817-824). Each pencil beam range pulling-back or reduction can be calculated by Rᵢ, where R_{Eo} is the range of the highest energy in water. Disclosed FLASH-RT Bragg peak treatment plans can employ a multi-field arrangement, for example a 5-field beam arrangement, and a multiple-field-optimization (MFO) method can be used to generate spot maps.

$$Equation 1$$

$${WET}_{i} ( x , y , z ) = {\int }_{0}^{depth} rsp ( x , y , z ) {dl}_{i} {R}_{i} = {R}_{E 0} - {WET}_{i} ( x , y , z ) \qquad ( 1 )$$

In embodiments, the total desired range compensation for each field can be achieved by using a URS and an RC. The thickness of the URS can vary, for example from 0 to a desired depth, which, with the assistance of the RC, enables the treatment of tumors at all depths.

In embodiments, the URS can comprise, for example, at least 1 plate, at least 2plates, at least 3 plates, at least 4 plates, at least 5 plates, at least 6 plates, at least 7 plates, at least 8 plates, at least 9 plates, at least 10 plates, at least 11 plates, at least 12 plates, at least 13 plates, at least 14 plates, at least 15 plates, at least 16 plates, at least 17 plates, at least 18 plates, at least 19 plates, at least 20 plates, or more In one example system, six plates may be used to generate a range of desired depths (0-34 cm) which may be adequate for many purposes.

In embodiments, the thickness of the individual URS plates can be, for example, 1 cm water equivalent thickness (WET), 2 cm WET, 3 cm WET, 4 cm WET, 5 cm WET, 6 cm WET, 7 cm WET, 8 cm WET, 9 cm WET, 10 cm WET, 11 cm WET, 12 cm WET, 13 cm WET, 14 cm WET, 15 cm WET, 16 cm WET, 17 cm WET, 18 cm WET, 19 cm WET, 20 cm WET, 21 cm WET, 22 cm WET, 23 cm WET, 24 cm WET, 25 cm WET, 26 cm WET, 27 cm WET, 28 cm WET, 29 cm WET, 30 cm WET, or more.

In an embodiment, the URS comprises 6 polycarbonate plastic plates of thicknesses of 1, 2, 3, 7, 7, and 14 cm WET, generating 35 discrete range reduction increments with a depth resolution of 1 cm. The range plate combinations for 35 discrete range pulling-backs used in the study described in Example 1 are depicted in Table 2.

FIG. 1(e) shows the schematic of a URS system of 6 polycarbonate plastic plates of thicknesses of 1, 2, 3, 7, 7, and 14 cm WET used in Example 1, generating 35 discrete range pulling-backs with a depth resolution of 1 cm. Each range shifter plate was driven by a standalone step motor to move “in” and “out” of the beam path, and the “in” and “out” combination of the six plates is similar to a binary system that can generate the correct range pulling back. The range plate combinations for 35 discrete range pulling-backs are depicted in Table 2.

In embodiments, the thicker range shifters can be placed closer downstream, and the thinner range shifters are more upstream, a design consideration to minimize the transport distance of scattered proton beams to reduce spot size and preserve a high spot peak dose rate (SPDR). The desired proton ranges are achieved by moving the range shifter plates “in” and “out” of the beam path. The thickness of URS used in each beam path can be calculated using R_{URS} in Eq.2. The max thickness of RC can be determined by R_{c} in Eq. 2. Therefore, in embodiments, the total range pulling-back capacity can be between 0 and R_{E0} cm which can accommodate deep and superficial targets.

The range compensation Ri of each proton trace under each field can be calculated and stored by, for example, a 3D data matrix. The data sets can be used to construct 3D printed compensators conveniently. As shown in FIG. 1(e), the RC is presented on the right upper and lower corner.

$$Equation 2$$

$$f ( x , y , z ) = max ( {WET}_{i} ( x , y , z ) ) - min ( {WET}_{i} ( x , y , z ) ) {R}_{C} = f cm and {R}_{URS} = ( {R}_{E 0} - f ) cm \qquad ( 2 )$$

Similar to the compensator design for scattering proton systems, a smearing method [Moyers M F, Miller D W, Bush D A, Slater J D. Methodologies and tools for proton beam design for lung tumors. Int J Radiat Oncol Biol Phys 2001; 49(5):1429-38.] can be used to design the RC to manage range uncertainties.

In embodiments, the minimal MU/spot or minimal treatment room beam current in nanoampere (nA) required for treatment can determine the dose rate of each energy layer, and the minimal MU/spot and dose rate are further optimized to reach the FLASH dose rate threshold. An algorithm can be used to generate an optimal spot map via two steps:
a. first, an initial minimal MU/spot threshold (w₀) can be used for inverse optimization and a dense spot map with a defined spot spacing can be generated;
b. second, the low weighting spots can be merged to new spots by applying both a distance threshold rₜ and a weighting factor of wₜ, in which the rₜ is a ratio of spot spacing. The wₜ is a factor-based minimal MU/spot requirement for FLASH dose rate. As shown in Eq.3, the weights can be combined as wₘ, and the spot coordinates are calculated based on their original coordinates and weighting fractions using Eq.4. The final spot location can be determined by applying the coordinate threshold rₜ, described using Eq. 5.

In some embodiments, there are at least two considerations for applying the second step to generate the final spot maps.
a. First, as the minimal MU/spot determines the SPDR of the layer, by merging the low weighting spots, a high SPDR can be achieved.
b. second, the low weighted spots are important to maintain a good plan quality as with conventional IMPT plan optimization. By merging lower weighted spots to the nearby ones, the spot distribution pattern is minimally changed, but better plan dosimetry distribution is achievable.

It will be appreciated that other planning steps and algorithms may also be employed.

In some disclosed embodiments, continuous optimization can be performed to fine-tune the spot weights to further improve target uniformity and OARs sparing. By iteratively applying the second step of spot map optimization (merging lower weighted spots to the nearby ones), the dose rate can be continuously improved.

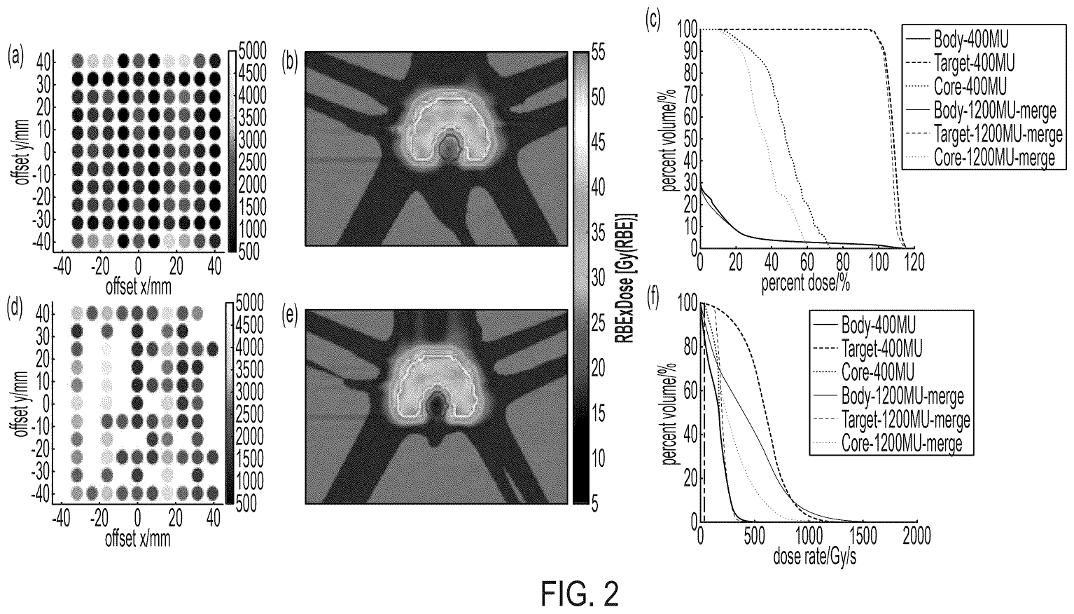

In embodiments, the efficacy of the spot map optimization can be tested using a C-shape target that surrounds a central avoidance core structure. As shown in FIG. 2, (a) is the spot map of one field using an initial 400 MU/spot threshold, (d) is the spot map after applying the spot map optimization process, (b) and (e) are the 2D dose distribution comparison for a selected slice, (c) and (f) are the DVH and DRVH comparison. By doing so, the low dose region is reduced, and the conformity improved, as can be seen from the 2D dose distribution, and the DVH of the core structure resultingly shifted towards the lower dose end substantially. The dose rates to body, target, and core structure can be increased, illustrated in (f).

$$Equations 3 , 4 , and 5$$

$${w}_{m} = {w}_{i} + {w}_{i + 1} \qquad ( 3 )$$

$$\vec{{r}_{m} ( x , y )} = \frac{{w}_{i}}{{w}_{m}} \vec{{r}_{i} ( x , y )} + \frac{{w}_{i + 1}}{{w}_{m}} \vec{{r}_{i + 1} ( x , y )} \qquad ( 4 )$$

$$\vec{{r}_{m} ( x , y )} = arg min \vert \vec{{r}_{m} ( x , y )} - \vec{{r}_{t} ( x , y )} \vert \qquad ( 5 )$$

FIG. 2 shows an example of spot distribution and weight optimization that can effectively improve the plan quality and FLASH-RT dose rate distribution. (a) and (d) the spot maps before and after the spot map optimization process; (b) and (e) the 2D dose distribution comparison; (c) and (f) the DVH and DRVH comparisons before and after spot map optimization. A dashed line from the DRVH marks the 40 Gy/s threshold.

In some embodiments, the multiple Coulomb scattering (MCS) between the protons and USR and RC can enlarge the spot divergence significantly. Equivalently, the scattering effects can also result in progressive shortening of the effective-SSD of the beam. At a shorter effective-SSD, proton fluence decreases more quickly due to a larger inverse square effect, and the spot size increases more rapidly.

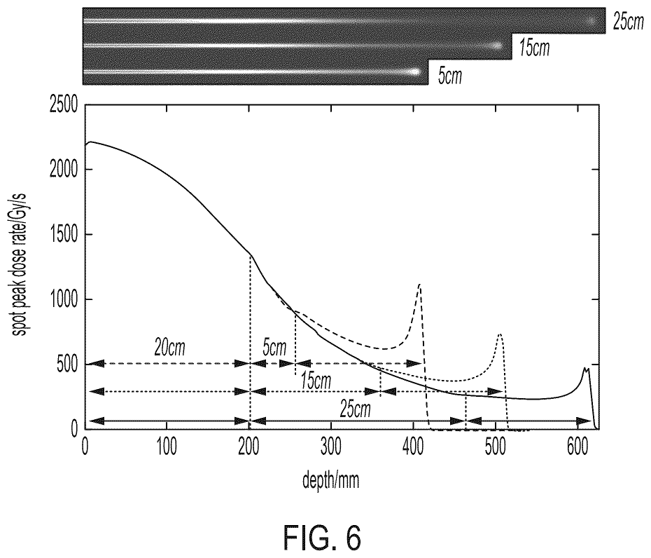

FIG. 6 illustrates the dose rate distribution for a 250 MeV single spot with 1000 Monitor Units (a measure of machine output from a clinical accelerator for radiation therapy; [MU]) in a water phantom. In Example 1, 5, 15, and 25 cm air gaps between the RC and the phantom surface were “mimicked” to calculate the spot dose rate at the central axis in water changing with air gaps. It was clear that the spot dose rate decreased when the air gap increased, and the central axis dose rate at the Bragg peak is reduced by a factor of ˜2 between 5 cm and 25 cm air gaps. During FLASH plan optimization, minimizing the air gap often plays an important role in maintaining proton fluence intensity and a smaller penumbra, which may be crucial for the OAR sparing. Meanwhile, a large spot size caused by MCS and a large air gap will significantly reduce the spot dose rate and the treatment field's mean dose rate. To achieve a higher spot dose rate, a relatively small air gap is critical for Bragg peak treatment planning.

FIG. 6. Upper view: the single spot (1000 MU/spot) 2D dose rate distribution for 250 MeV proton beam at central axis plane evolves in water phantom with air gaps of 5, 15, and 25 cm, respectively; lower view: the spot dose rate at the central axis (the three sections represent the 20 cm transport in water, air gaps, and the residual range in water).

Disclosed embodiments can produce field dose rates of, for example, at least 1 Gy/s, or more. For example, in embodiments, the dose rate is at least 5 Gy/s, at least 10 Gy/s, at least 15 Gy/s, at least 20 Gy/s, at least 25 Gy/s, at least 30 Gy/s, at least 35 Gy/s, at least 42 Gy/s, at least 44 Gy/s, at least 46 Gy/s, at least 48 Gy/s, at least 50 Gy/s, at least 52 Gy/s, at least 56 Gy/s, at least 58 Gy/s, at least 60 Gy/s, at least 62 Gy/s, at least 64 Gy/s, at least 66 Gy/s, at least 68 Gy/s, at least 70 Gy/s, at least 72 Gy/s, at least 74 Gy/s, at least 76 Gy/s, at least 78 Gy/s, at least 80 Gy/s, at least 82 Gy/s, at least 84 Gy/s, at least 86 Gy/s, at least 88 Gy/s, at least 90 Gy/s, at least 92 Gy/s, at least 94 Gy/s, at least 96 Gy/s, at least 98 Gy/s, at least 100 Gy/s, at least 102 Gy/s, at least 104 Gy/s, at least 106 Gy/s, at least 108 Gy/s, at least 110 Gy/s, at least 112 Gy/s, at least 114 Gy/s, at least 116 Gy/s, at least 118 Gy/s, at least 120 Gy/s, at least 122 Gy/s, at least 124 Gy/s, at least 126 Gy/s, at least 128 Gy/s, at least 130 Gy/s, at least 132 Gy/s,or more.

Disclosed embodiments further comprise computer-readable instructions for determining the employment of the range shifters and compensators. For example, in disclosed embodiments, computer-readable instructions can comprise instructions for calculating the number, thickness, and placement of URS and RCs based upon the characteristics of the non-shifted and non-compensated ionizing radiation, the target treatment depth, and the target's three-dimensional shape.

In embodiments, disclosed systems do not comprise quadrupole magnets.

In embodiments, after reaching the treatment nozzle, the range of the highest-energy proton beams is tailored using a range pullback mechanism comprising a URS and a RC. In embodiments the URS can be flexibly assembled from buildup materials of various thicknesses, such as solid water or plastics. In embodiments the RC consists of the remaining buildup materials needed to adjust the proton Bragg peaks to match the distal edge of the target.

3D printing can facilitate such irregular-shaped RCs. In order to achieve IMPT delivery, the plans will be delivered with multiple field angles in a similar fashion to the use of conventional multi-energy proton beams. In embodiments, the specific combination of RC and URS for each beam will be determined by the in-house developed treatment planning system (TPS).

### Methods of Use

Methods disclosed herein can comprise producing shifted and compensated ionizing radiation from an initial ionizing radiation source. For example, in embodiments, the initial ionizing radiation source can comprise protons, a-rays, carbon ions, other ion rays, and combinations thereof. In embodiments, methods comprise the use of a cyclotron or a synchrotron to produce an ionizing radiation, which is then subject to range shifter and range compensation.

Disclosed embodiments comprise targeting the ionizing radiation. In embodiments, the ionizing radiation is targeted to a point or area within the target tissue plus an expansion margin, for example between the center of the target tissue and a point within 5 cm beyond (extending outwardly from the center) the edge of the target tissue. For example, in embodiments, the ionizing radiation is targeted so that the Bragg peak of the ionizing radiation coincides with a point within or near the perimeter or margin of the target tissue, such as a distal (with respect to the ionizing radiation transport direction) edge, a proximal edge, or a lateral edge of the target tissue. In embodiments, disclosed methods comprise proton arc therapy wherein the proton beam is administered from multiple angles relative to the patient. For example, in embodiments, the proton beam is administered from at least 2, 3, 4, 5, 6, 7, 8, 9, 10, 11, 12, 13, 14, 15, 16, 17, 18, 19, or 20 or more angles.

For example, in embodiments the ionizing radiation is targeted to a distance from perimeter/margin of the target tissue, such as an edge of a tumor. For example, in embodiments, the ionizing radiation is targeted 1 mm from an edge of the target tissue, or 2 mm from an edge of the target tissue, or 3 mm from an edge of the target tissue, or 4 mm from an edge of the target tissue, or 5 mm from an edge of the target tissue, or 6 mm from an edge of the target tissue, or 7 mm from an edge of the target tissue, or 8 mm from an edge of the target tissue, or 9 mm from an edge of the target tissue, or 10 mm from an edge of the target tissue, or 11 mm from an edge of the target tissue, or 12 mm from an edge of the target tissue, or 13 mm from an edge of the target tissue, or 14 mm from an edge of the target tissue, or 15 mm from an edge of the target tissue, or 16 mm from an edge of the target tissue, or 17 mm from an edge of the target tissue, or 18 mm from an edge of the target tissue, or 19 mm of an edge of the target tissue, or 20 mm of an edge of the target tissue, or more.

For example, in embodiments the ionizing radiation is targeted to or around the perimeter/margin of the target tissue, such as an edge of a tumor. For example, in embodiments, the ionizing radiation is targeted to within 1 mm of an edge of the target tissue, or within 2 mm of an edge of the target tissue, or within 3 mm of an edge of the target tissue, or within 4 mm of an edge of the target tissue, or within 5 mm of an edge of the target tissue, or within 6 mm of an edge of the target tissue, or within 7 mm of an edge of the target tissue, or within 8 mm of an edge of the target tissue, or within 9 mm of an edge of the target tissue, or within 10 mm of an edge of the target tissue, or within 11 mm of an edge of the target tissue, or within 12 mm of an edge of the target tissue, or within 13 mm of an edge of the target tissue, or within 14 mm of an edge of the target tissue, or within 15 mm of an edge of the target tissue, or within 16 mm of an edge of the target tissue, or within 17 mm of an edge of the target tissue, or within 18 mm of an edge of the target tissue, or within 19 mm of an edge of the target tissue, or within 20 mm of an edge of the target tissue, or more.

In embodiments, the ionizing radiation is targeted toward the center of a tumor, or within a distance on either side from the center. For example, in embodiments, the ionizing radiation is targeted to within 1 mm of the center of the target tissue, or within 2 mm of the center of the target tissue, or within 3 mm of the center of the target tissue, or within 4 mm of the center of the target tissue, or within 5 mm of the center of the target tissue, or within 6 mm of the center of the target tissue, or within 7 mm of the center of the target tissue, or within 8 mm of the center of the target tissue, or within 9 mm of the center of the target tissue, or within 10 mm of the center of the target tissue, or within 11 mm of the center of the target tissue, or within 12 mm of the center of the target tissue, or within 13 mm of the center of the target tissue, or within 14 mm of the center of the target tissue, or within 15 mm of the center of the target tissue, or within 16 mm of the center of the target tissue, or within 17 mm of the center of the target tissue, or within 18 mm of the center of the target tissue, or within 19 mm of the center of the target tissue, or within 20 mm of the center of the target tissue, or more.

In further embodiments, the ionizing radiation is targeted to a point between an edge of the target tissue and the three-dimensional center of the target tissue.

In embodiments, range shifting comprises the use of multiple plastic plates, for example transparent amorphous thermoplastic plates such as polycarbonate plastic plates, with varying thicknesses, to reduce the range of the initial ionizing radiation to the desired range. For example, in embodiments, an inverse-planning tool is used to determine the number, position, and thickness of the individual URS plates necessary to lower the range of the initial ionizing radiation to the desired range.

In embodiments, disclosed methods can comprise diagnosis of a cancerous tissue, for example by the use of lab tests, imaging tests, biopsy, and the like. For example, elevated or depressed levels of certain substances in the body can be a sign of cancer. Therefore, lab tests of blood, urine, or other body fluids or cells can measure these substances and help doctors make a diagnosis. In embodiments, lab tests involve testing blood or tissue samples for tumor markers. Tumor markers are substances that are produced by cancer cells or by other cells of the body in response to cancer. Most tumor markers are made by normal cells and cancer cells, but they are generally produced at much higher levels by cancer cells.

Disclosed methods can further comprise the use of imaging tests to identify cancerous tissue. For example, imaging tests visualize areas inside the body that help identify cancerous tissue. Disclosed methods comprising imaging tests can comprise, for example, CT scans, wherein an x-ray machine linked to a computer takes a series of pictures of internal organs from different angles. These pictures are used to create detailed 3-D images of the inside of the body.

Further methods comprise use of magnetic resonance imaging (MRI). An MRI uses a powerful magnet and radio waves to take pictures of the body in sections, which can show the difference between healthy and unhealthy tissue.

Further disclosed methods can comprise nuclear scanning, which uses uses radioactive material to image the inside of the body. This type of scan may also be called radionuclide scan.

Additional disclosed methods of treatment can comprise bone scanning, which is a type of nuclear scan that is used to identify anomalies in bone.

Further disclosed methods can comprise a positron emission tomography (PET) scan. A PET scan is an imaging test that allows your doctor to check for diseases in your body. The scan uses a special dye containing radioactive tracers. These tracers are either swallowed, inhaled, or injected into a vein in a patient's arm depending on what part of the body is being examined.

Further diagnostic methods suitable for use in disclosed embodiments can comprise ultrasound. An ultrasound exam uses high-energy sound waves that “echo” off tissues inside the body. A computer uses these echoes to create pictures of areas inside your body.

In embodiments, multiple diagnostic methods can be used to identify prospective areas of treatment. In embodiments, the area of treatment comprises a tumor, for example malignant tumors.

Once diagnosed, the area of treatment can be mapped and a treatment plan developed. For example, after a target tissue is three-dimensionally mapped, appropriate URS, RC, dosages, and beam angles are determined.

The treatment plan can then be applied as administration of range shifted and compensated ionizing radiation at an appropriate dose. For example, disclosed methods can comprise treatment dose of, for example, at least 1.8 Gy/fraction, or more. For example, in embodiments, the dose is at least 1.8 Gy/fraction, at least 2 Gy/fraction, at least 3 Gy/fraction, at least 4 Gy/fraction, at least 5 Gy/fraction, at least 6 Gy/fraction, at least 8 Gy/fraction, at least 10 Gy/fraction, at least 12 Gy/fraction, at least 14 Gy/fraction, at least 16 Gy/fraction, at least 18 Gy/fraction, at least 20 Gy/fraction, at least 22 Gy/fraction, at least 24 Gy/fraction, at least 26 Gy/fraction, at least 28 Gy/fraction, at least 30 Gy/fraction, at least 32 Gy/fraction, at least 34 Gy/fraction, at least 36 Gy/fraction, at least 38 Gy/fraction, at least 40 Gy/fraction, at least 42 Gy/fraction, at least 44 Gy/fraction, at least 46 Gy/fraction, at least 48 Gy/fraction, at least 50 Gy/fraction, at least 52 Gy/fraction, at least 54 Gy/fraction, at least 56 Gy/fraction, at least 58 Gy/fraction, at least 60 Gy/fraction, at least 62 Gy/fraction, at least 64 Gy/fraction, at least 66 Gy/fraction, at least 68 Gy/fraction, at least 70 Gy/fraction, or more.

In embodiments, the fraction dose is, for example, 1.8 Gy/fraction, or more. For example, in embodiments, the dose is 1.8 Gy/fraction, 2 Gy/fraction, 3 Gy/fraction, 4 Gy/fraction, 5 Gy/fraction, 6 Gy/fraction, 8 Gy/fraction, 10 Gy/fraction, 12 Gy/fraction, 14 Gy/fraction, 16 Gy/fraction, 18 Gy/fraction, 20 Gy/fraction, 22 Gy/fraction, 24 Gy/fraction, 26 Gy/fraction, 28 Gy/fraction, 30 Gy/fraction, 32 Gy/fraction, 34 Gy/fraction, 36 Gy/fraction, 38 Gy/fraction, 40 Gy/fraction, 42 Gy/fraction, 44 Gy/fraction, 46 Gy/fraction, 48 Gy/fraction, 50 Gy/fraction, 52 Gy/fraction, 54 Gy/fraction, 56 Gy/fraction, 58 Gy/fraction, 60 Gy/fraction, 62 Gy/fraction, 64 Gy/fraction, 66 Gy/fraction, 68 Gy/fraction, 70 Gy/fraction, or the like.

In embodiments, the fraction dose is not more than 1.8 Gy/fraction, not more than 2 Gy/fraction, not more than 3 Gy/fraction, not more than 4 Gy/fraction, not more than 5 Gy/fraction, not more than 6 Gy/fraction, not more than 8 Gy/fraction, not more than 10 Gy/fraction, not more than 12 Gy/fraction, not more than 14 Gy/fraction, not more than 16 Gy/fraction, not more than 18 Gy/fraction, not more than 20 Gy/fraction, not more than 22 Gy/fraction, not more than 24 Gy/fraction, not more than 26 Gy/fraction, not more than 28 Gy/fraction, not more than 30 Gy/fraction, not more than 32 Gy/fraction, not more than 34Gy/fraction, not more than 36 Gy/fraction, not more than 38 Gy/fraction, not more than 40 Gy/fraction, not more than 42 Gy/fraction, not more than 44 Gy/fraction, not more than 46 Gy/fraction, not more than 48 Gy/fraction, not more than 50 Gy/fraction, not more than 52 Gy/fraction, not more than 54 Gy/fraction, not more than 56 Gy/fraction, not more than 58 Gy/fraction, not more than 60 Gy/fraction, not more than 62 Gy/fraction, not more than 64 Gy/fraction, not more than 66 Gy/fraction, not more than 68 Gy/fraction, not more than 70 Gy/fraction, or the like.

In embodiments, the range shifted and compensated ionizing radiation is administered in multiple fields. For example, in embodiments, 2 fields are administered, or 3, or 4, or 5, or 6, or 7, or 8, or 9, or the like. In embodiments, at least 2 fields are administered, or at least 3, or at least 4, or at least 5, or at least 6, or at least 7, or at least 8, or at least 9, or the like. In embodiments, not more than 2 fields are administered, or not more than 3, or not more than 4, or not more than 5, or not more than 6, or not more than 7, or not more than 8, not more than 9, not more than 10, not more than 11, not more than 12, not more than 13, not more than 14, not more than 15, or the like.

In embodiments, the angles at which the fields of range shifted and compensated ionizing radiation are administered to the target tissue are equivalent between the fields. For example, in embodiments wherein 5 fields are administered, the angles at which the fields are administered can be 72 degrees apart. The angle between the fields can be determined as appropriate to meet the clinical requirement.

Further embodiments comprise treatment with at least one chemotherapeutic agent.

The frequency of employment of the disclosed methods can be determined based on the nature and location of the particular area being treated. In certain cases, however, repeated treatment in the future may be desired to achieve optimal results.

All of the disclosed methods and procedures described in this disclosure can be implemented using one or more computer programs or components. These components may be provided as a series of computer instructions on any conventional computer readable medium or machine readable medium, including volatile and non-volatile memory, such as RAM, ROM, flash memory, magnetic or optical disks, optical memory, or other storage media. The instructions may be provided as software or firmware, and may be implemented in whole or in part in hardware components such as ASICs, FPGAS, DSPs, or any other similar devices. The instructions may be configured to be executed by one or more processors, which when executing the series of computer instructions, performs or facilitates the performance of all or part of the disclosed methods and procedures.

### EXAMPLES

The following non-limiting Examples are provided for illustrative purposes only in order to facilitate a more complete understanding of representative embodiments. This example should not be construed to limit any of the embodiments described in the present specification.

### Example 1

### Treatment of Lung Tumors

Using the single-energy beam of the cyclotron or synchrotron machine, the Bragg peak plan achieved sufficient beam current for FLASH dose rate; meanwhile, multiple-field inverse optimization treatment planning with range pulling-back devices (URS) in the beam path enabled the treatment planning to eliminate the exit dose beyond targets. once the treatment plan meets the target coverage and OARs constraints, a raytracing method created a range compensator for each field. As shown in FIG. 1(e), a 5-field IMPT plan used a universal range shifter (URS) and range compensators (RC) for lung cancer FLASH-RT, and the proton range was tailored to adapt to the target distal edge.

FIG. 1. Schematic diagram of non-transmission FLASH IMPT planning used a universal range shifter (URS) and range compensators (RC). The URS and RC were placed in the beam path for illustration purpose only. (a) A phantom example that used a 6-mm distal margin to determine the location of the proton stoppage. The dots represent where the protons stop, and the integrated water equivalent thickness (WET) distance from the body contour was calculated to determine the range pulling-back and compensator contour; (b) dose distribution using a single-energy layer based on the spot map of (a); (c) beam-eye-view of 2D range compensation calculated by a ray-tracing method; (d) the 1D range compensation at the central axis; (e) a 5-field beam arrangement for a lung treatment plan, with the upper and lower corner illustrating one of the 5-RC with two different views.

### Inverse Planning to Achieve IMPT Treatment Plans

Ray tracing to Calculate the Range Compensation

A single-energy beam was customized to generate the intensity-modulated spot map via the inverse planning platform. As shown in FIG. 1 (a), a uniform margin of 6-mm on the CTV was used to contain the spot distribution in-depth direction. The 90% of dose falloff was used as the proton range for spot map generation. The water equivalent thickness (WET) of each pencil beam proton radiographic track (denoted by WETi (x, y, z)) was calculated by Eq.1, and rsp(x, y, z) represents the relative stopping power (rsp) of each voxel of the 3D CT images. The integral step in Eq.1 was accurately computed with a raytracing algorithm. Each pencil beam range pulling-back is calculated by Rᵢ, where R_{Eo} is the range of the highest energy in water. All FLASH-RT Bragg peak plans used a 5-field beam arrangement, and an MFO method was used to generate spot maps.

$$Equation 1$$

$${WET}_{i} ( x , y , z ) = {\int }_{0}^{depth} rsp ( x , y , z ) {dl}_{i} {R}_{i} = {R}_{E 0} - {WET}_{i} ( x , y , z ) \qquad ( 1 )$$

### Universal Range Shifter (URS) and Range Compensator (RC)

The total range compensation for each field was achieved by using a URS and an RC. The thickness of a URS changes from 0 to the desired thickness, with the assistance of RC, enables the treatment of tumors at all depths.

The URS consisted of 6 polycarbonate plastic plates of thicknesses of 1, 2, 3, 7, 7, and 14 cm WET, generating 35 discrete range pulling-backs with a depth resolution of 1 cm. Each range shifter plate was driven by a standalone step motor to move “in” and “out” of the beam path, and the “in” and “out” combination of the six plates is similar to a binary system that can generate the correct range pulling back. The range plate combinations for 35 discrete range pulling-backs are depicted in Table 2.

FIG. 1(e) shows the schematic of the URS system, and the thicker range shifters are placed closer downstream. The thinner range shifters were more upstream, a design consideration to minimize the transport distance of scattered proton beams to reduce spot size and preserve a high SPDR. The desired proton ranges were achieved by moving the range shifter plates “in” and “out” of the beam path. The thickness of URS used in each beam path was be calculated using R_{URS} in Eq.2. The max thickness of R_{C} is determined by R_{C} in Eq. 2. Therefore, the total range pulling-back capacity is between 0 and R_{E0} cm that can accommodate the deepest targets to the superficial targets. The range compensation Ri of each proton trace under each field is calculated and stored by a 3D data matrix. The data sets can be used to construct 3D printed compensators conveniently. As shown in FIG. 1(e), the RC is presented on the right upper and lower corner.

$$Equation 2$$

$$f ( x , y , z ) = max ( {WET}_{i} ( x , y , z ) ) - min ( {WET}_{i} ( x , y , z ) ) {R}_{C} = fcm and {R}_{URS} = ( {R}_{E 0} - f ) cm \qquad ( 2 )$$

Similar to the compensator design for scattering proton systems, a smearing method [Moyers M F, Miller D W, Bush D A, Slater J D. Methodologies and tools for proton beam design for lung tumors. Int J Radiat Oncol Biol Phys 2001; 49(5):1429-38.] was used to design the RC to manage range uncertainties. A robust plan is important to achieve the clinical intention for the target and OARs. The major uncertainties resulting from setup and motion can cause large range uncertainties. A 3.5% CT Hounsfield unit (HU) to relative stopping conversion uncertainties and 3-mm setup errors were used when applying the smearing method.

### Spot Map Optimization to Improve Plan Quality and Dose Rate

Next, the minimum MU/spot or minimum treatment room beam current in nanoampere (nA) determined the dose rate of each energy layer, and the minimal MU/spot and dose rate needs to be further optimized to reach the FLASH dose rate threshold. An in-house algorithm was developed to generate an optimal spot map via two steps: first, an initial minimum MU/spot threshold (w₀) was used for inverse optimization and a dense spot map with a defined spot spacing was generated; second, the low weighting spots were merged to new spots by applying both a distance threshold n and a weighting factor of wₜ, in which the rₜ is a ratio of spot spacing. The wi is a factor-based minimum MU/spot requirement for FLASH dose rate. As shown in Eq.3, the weights are combined as wₘ, and the spot coordinates are calculated based on their original coordinates and weighting fractions using Eq.4. The final spot location is determined by applying the coordinate threshold rₜ, described using Eq. 5. In general, there are two considerations for applying the second step to generate the final spot maps. First, as the minimum MU/spot determines the SPDR of the layer, by merging the low weighting spots, a high SPDR can be achieved. Second, the low weighted spots significantly contribute to maintaining a good plan quality as with conventional IMPT plan optimization. By merging lower weighted spots to the nearby ones, the spot distribution pattern is minimally changed, but better plan dosimetry distribution is achievable.

Continuous optimization was performed to fine-tune the spot weights to further improve target uniformity and OARs sparing. By iteratively applying the second step of spot map optimization, the dose rate was continuously improved. The efficacy of the spot map optimization was tested using a C-shape target that surrounds a central avoidance core structure. As shown in FIG. 2, (a) is the spot map of one field using an initial 400 MU/spot threshold, (d) is the spot map after applying the spot map optimization process, (b) and (e) are the 2D dose distribution comparison for a selected slice, (c) and (f) are the DVH and DRVH comparison. It was evident that the low dose region was reduced, and the conformity was improved, as can be seen from the 2D dose distribution, and the DVH of the core structure resultingly shifted towards the lower dose end substantially. The dose rates to body, target, and core structure were increased, illustrated in (f).

$${w}_{m} = {w}_{i} + {w}_{i + 1} \qquad ( 3 )$$

$$\vec{{r}_{m} ( x , y )} = \frac{{w}_{i}}{{w}_{m}} \vec{{r}_{i} ( x , y )} + \frac{{w}_{i + 1}}{{w}_{m}} \vec{{r}_{i + 1} ( x , y )} \qquad ( 4 )$$

$$\vec{{r}_{m} ( x , y )} = arg min \vert \vec{{r}_{m} ( x , y )} - \vec{{r}_{t} ( x , y )} \vert \qquad ( 5 )$$

FIG. 2 shows an example of spot distribution and weight optimization that can effectively improve the plan quality and FLASH-RT dose rate distribution. (a) and (d) the spot maps before and after the spot map optimization process; (b) and (e) the 2D dose distribution comparison; (c) and (f) the DVH and DRVH comparisons before and after spot map optimization. A dashed line from the DRVH marks the 40 Gy/s threshold.

### Spot Dose Rate Evolution in Range Shifter, Air, and Phantom

The MCS between the protons and USR and RC can enlarge the spot divergence significantly. Equivalently, the scattering effects have also resulted in progressive shortening of the effective-SSD of the beam. At a shorter effective-SSD, proton fluence decreases more quickly due to a larger inverse square effect, and the spot size increases more rapidly. FIG. 6 illustrates the dose rate distribution for a 250 MeV single spot with 1000 MU in a water phantom. We mimicked 5, 15, and 25 cm air gaps between the RC and the phantom surface to calculate the spot dose rate at the central axis in water changing with air gaps. It was clear that spot dose rate decreased when the air gap increased, and the central axis dose rate at the Bragg peak is reduced by a factor of ˜2 between 5 cm and 25 cm air gaps. During FLASH plan optimization, minimizing the air gap plays an important role in maintaining proton fluence intensity and a smaller penumbra, which is crucial for the OAR sparing. Meanwhile, a large spot size caused by MCS and a large air gap will significantly reduce the spot dose rate and the treatment field's mean dose rate. To achieve a higher spot dose rate, a relatively small air gap is critical for Bragg peak treatment planning.

FIG. 6. Upper view: the single spot (1000MU/spot) 2D dose rate distribution for 250 MeV proton beam at central axis plane evolves in water phantom with air gaps of 5, 15, and 25 cm, respectively; lower view: the spot dose rate at the central axis (the three sections represent the 20 cm transport in water, air gaps, and the residual range in water).

### Feasibility Study using Both Phantom and Patient Images

A rectangular water “phantom” with a C-shape target was used for planning to investigate the dosimetry quality and dose rate distribution for both transmission and Bragg peak plans. At the same time, six consecutive lung cancer patients previously treated with proton SBRT at our facility were re-planned to receive both transmission and Bragg peak FLASH plans to assess the dose and dose rate distribution. The planning goal of using Bragg peak FLASH-RT was to achieve a comparable V_{40Gy/s} dose rate coverage for critical OARs but substantially improved OAR sparing while using OAR dose constraints from the original clinical treatment parameters. Marlen et al. reported a FLASH transmission study using 8 to 12 non-coplanar beams for lung cancer planning. The reason to use more fields was to reduce normal lung dose and improve the target conformality. To make a “fair” dosimetry comparison, this study used 5-field plans with even angle separation (72 degrees) for both transmission and Bragg peak plans. The phantom plan used a prescription of 50 Gy in 1 fraction, and the patient plans used a standard-of-care prescription of 34 Gy in 1 fraction. The minimal MU to maintain the FLASH dose rate for the transmission and Bragg peak plans was 400 MU/spot and 1200 MU/spot, respectively. The 3D dose rate quantification used the DADR method, and DRVHs are investigated for both phantom and patient plans.

### Results

Bragg Peak FLASH-RT Planning using Phantom

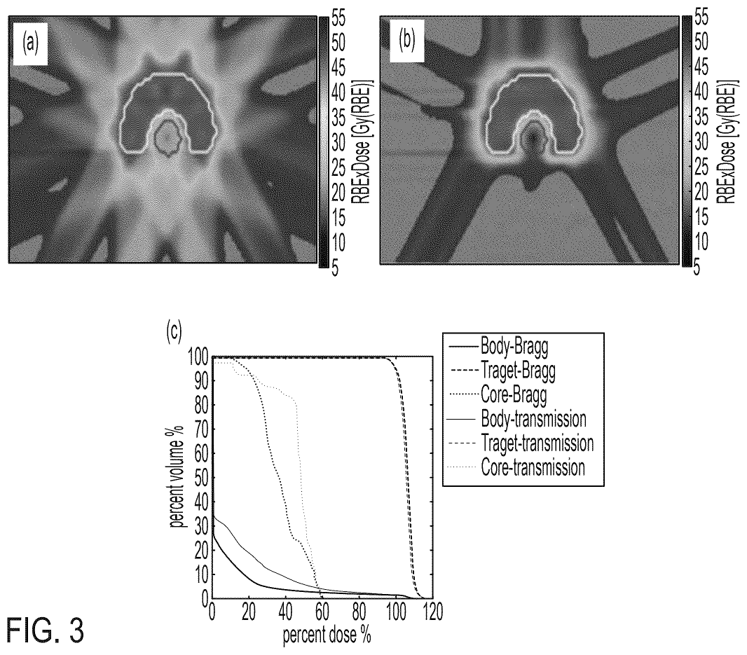

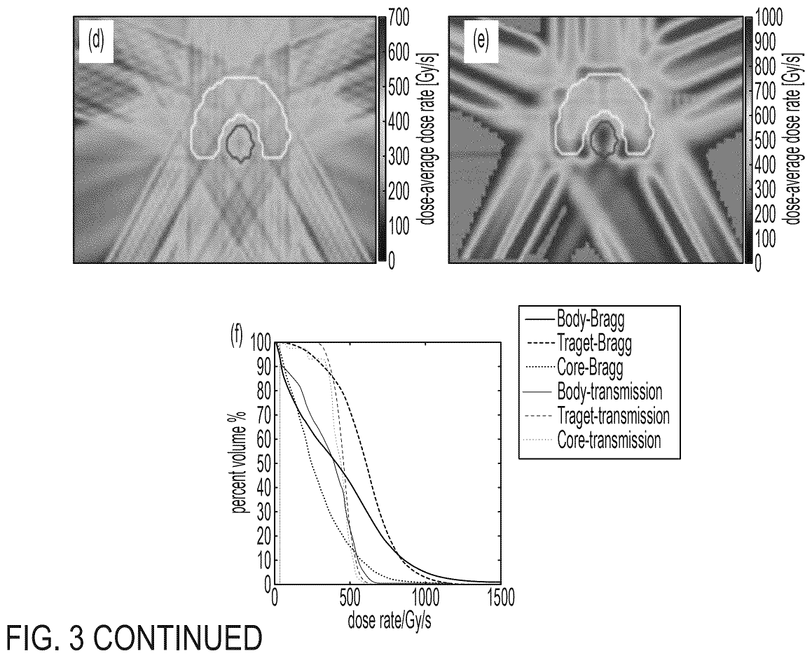

The phantom planning used five fields with 72 degrees equal angle separation, and the distance between the core and C-shape target was about 1.5 cm. FIG. 3 (a) and (b) are the 2D dose distribution for a selected slice. The Bragg peak plan resulted in less low dose scattering cloud and integral dose than the transmission plans due to the non-existence of exit dose with the Bragg peak method. The target coverage and uniformity were nearly identical but with much less dose spillage to the body and core structures. As shown in FIG. 3(c), the DVHs of the body and core had a large separation between the two FLASH delivery methods, demonstrating that the Bragg peak plans reduce dose spillage from low to a medium level significantly. FIG. 3(d) and (e) are the DADR dose rate distribution for the same image slice, and FIG. 3(f) is the DRVH comparison. It was evident that the transmission plans tended to generate a higher dose rate distribution versus Bragg peak plans. However, Bragg peak can also reach FLASH-RT threshold 40_{Gy/s} by increasing the minimal MU/spot (1200 MU/spot) and via dose rate optimization. As illustrated in FIG. 3(f), after applying the spot map optimization, the V_{40Gy/s} coverage of the body and surrogate OAR core structure was as high as 95%. The phantom dosimetry and dose rate comparison indicated that the Bragg peak plans can achieve similar target coverage, but much better OAR sparing compared to the transmission plans. At the same time, the FLASH-RT dose rate can be successfully maintained by the Bragg peak plan.

FIG. 3. Transmission ((a) & (d)) vs. Bragg peak ((b) & (e)) planning using 250 MeV proton beams for C-shape target in a water phantom: (a) and (b) are the 2D dose distribution for a selected slice, (c) is the DVH comparison between them; (d) and (e) are the 2D dose rate distribution, and (f) is the DRVH comparison. A dashed line from the DRVH marks the 40_{Gy/s} threshold.

### Lung HYPOFRACTIONATION Plan Study

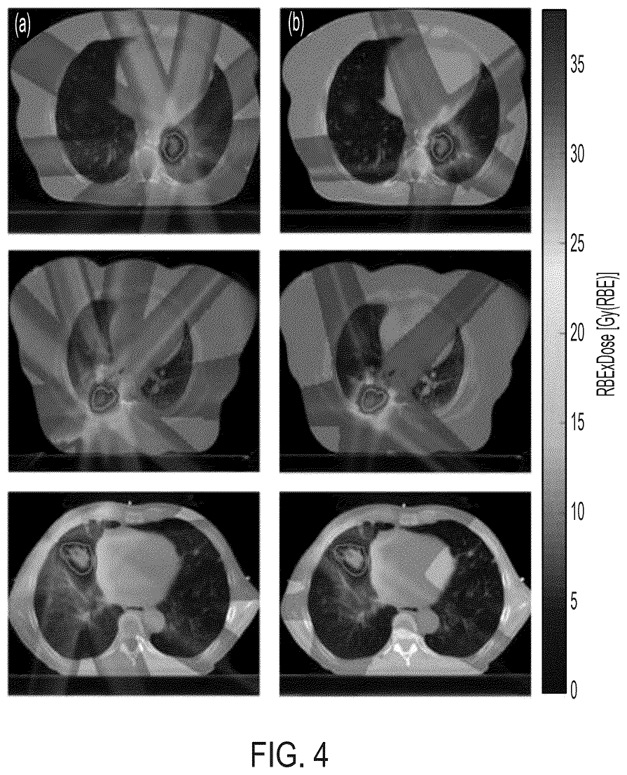

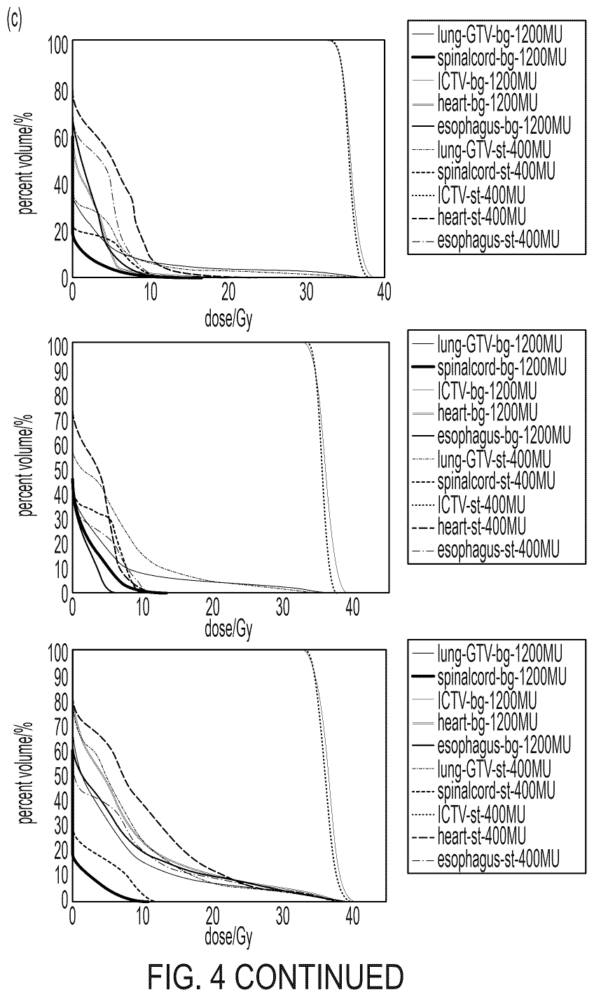

The 2D dose distribution is displayed in FIG. 4(a) and FIG. 4(b) for the three selected cases. As illustrated from the dose color wash, the Bragg peak plans were superior in low and medium dose regions. The scattering dose cloud and integral dose were significantly less in dose distributions of Bragg peak plans for all three cases. The 3-DVH (FIG. 4(c)) also showed a significant dose-volume reduction for lung-GTV, esophagus, spinal cord, and heart when using the Bragg peak method for FLASH planning. Table 1 compiles the dosimetry parameters based on the RTOG0915 protocol for both transmission and Bragg peak IMPT plans [RTOG0915. https://www.nrgoncology.org/Clinical-Trials/Protocol/rtog-0915?filter=rtog-0915] for all six lung patients. D2% represents the high dose region and dose uniformity of the target. The Bragg peak plans yielded slightly worse uniformity than the transmission plan (110.6% vs. 112.2% of the prescription dose) due to the increase of minimum MU/spot. Most of the OAR dose metrics, however, are superior for Bragg peak plans. For instance, for lung-GTV, the mean volume of V7Gy is reduced from 724.9 cc to 492.6 cc, which corresponds to a volume reduction up to 32% (p=0.001); the mean volume of V7.4Gy is reduced from 672.8 cc to 468.7 cc with an irradiated volume reduction of 30% (p=0.002). Additionally, while the doses to the esophagus, heart, and spinal cord are sensitive to the target locations and beam arrangements, some of those OARs could be completely spared in Bragg peak plans if there were no beam passing through or toward them. Given the larger statistical errors and the heterogeneity of tumor sites in this initial cohort, these differences were not statistically significant (p>0.05).

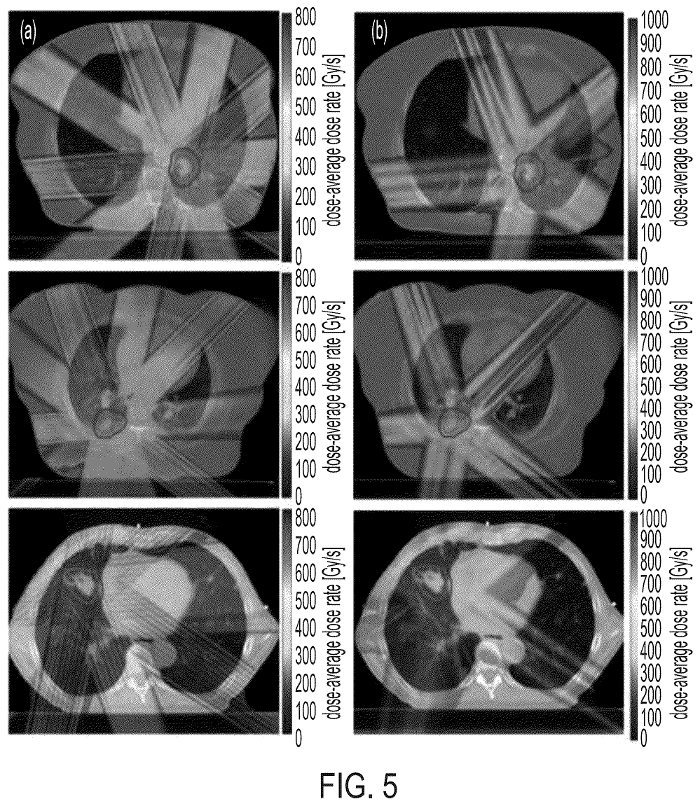

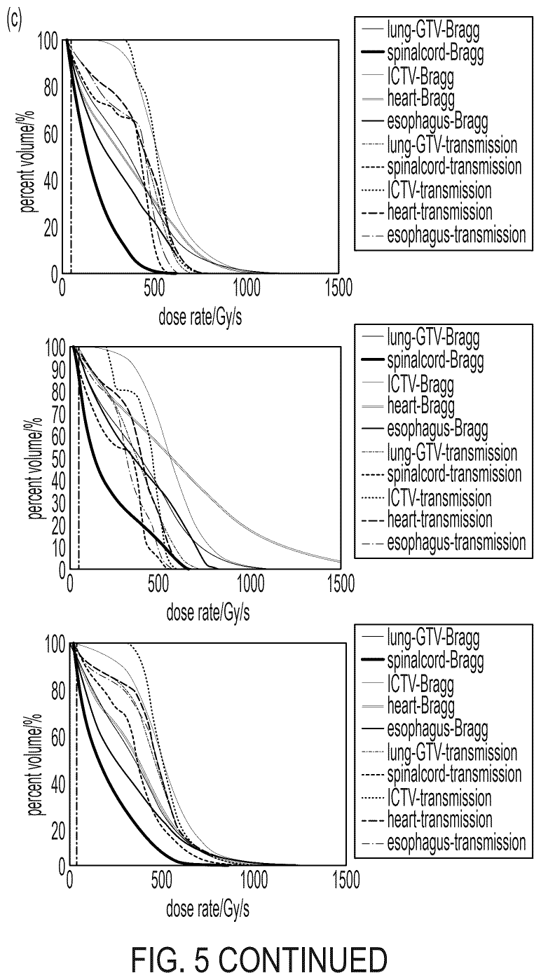

FIG. 5 is the 2D dose rate distribution and DRVH comparison for the three selected lung cancer patients. The left column shows the dose rate distribution of the transmission plan, illustrating that the proton dose rate attenuates with the depth when passing through tissues, and the exit dose rate is much lower than the entrance dose rate for each of the single fields. The middle column is the dose rate distribution for Bragg peak plans with high dose rate strips and low dose rate valleys observed in each of the fields. As is known, the freedom of plan optimization includes spot maps, spot weights, number of spots and fields, and minimal MU/spot, etc. Using 400 MU/spot and 1200 MU/spot for transmission and Bragg peak plans, the increase of minimum MU/spot will increase the difficulty to achieve good uniformity and OARs sparing for the Bragg peak plan. All parameters are optimized except for energy and the number of fields to fulfill the required dosimetry objects via inverse optimization. Here the highly modulated fluence serves as compensation for reducing flexibility with increasing MU/spot in maintaining a higher 3D dose rate. The inverse algorithm plays a crucial role in achieving a uniform dose distribution via non-uniform dose fluence by the MFO method. The V_{40Gy/s} dose rate coverage was compiled in Table 1. All targets can reach almost 100% V_{40Gy/s}, and the mean V_{40Gy/s} coverage of all OARs is at least >91.0+3.8% (spinal cord of Bragg peak plans) for both transmission and Bragg peak plans. In all, the dose distribution, DVHs, and dosimetry metrics comparison demonstrate substantially improved sparing for lung, cord, heart, and esophagus with Bragg peak plans; in contrast, those plans preserved the FLASH dose rate.

Table 1. Dosimetry and dose rate coverage of V_{40Gy/s} comparison for transmission and Bragg peak IMPT plans for all six lung cases. The dosimetry comparison used RTOG 0915 metrics. Both dose and dose rate statistics used the averaged values for all six cases. The last row of the table represents the averaged V_{40Gy/s} for both target and OARs.

FIG. 4. The dose comparisons between transmission and Bragg peak plans for three selected lung patients using the same beam arrangement. The right and middle columns represent transmission and Bragg peak plans, respectively. (a) and (b) 2D dose distribution for selected slices, (c) DVH comparison between the two types of plans.

FIG. 5. Dose rate comparisons between transmission (the left side images) and Bragg peak (the middle images) plans using the same beam arrangement. (a) and (b) 2D dose rate distribution for selected slices, (c) DRVH comparison between the two types of plans. A dashed line marks the 40_{Gy/s} threshold.

### Discussion

While the MU definition varied between different proton vendors, the universal quantification for dose rate is to use the beam current at the treatment room. However, in treatment planning, MU is used as a basic unit to calculate the planned dose. To describe the dose rate precisely, the correlation between beam current and the number of protons per MU needs to be determined, e.g., by Monte Carlo simulation or experimental methods. The treatment planning system (TPS) capability to optimize the spot weight may vary between different TPSs. Different optimizers and dose calculation engines can result in varying plan qualities and dose rate distributions. Therefore, treatment planning strategies, the DVH, and DRVH distribution need to be extensively studied as these commercial TPSs are available for clinical application. Due to the intrinsic nature of dose rate attenuation when PBS spots pass through URS, RC, air gap, and patient tissue, the 3D dose rate distribution for OARs is a function of proton range pulling-back and minimal MU/spot. To treat shallow targets, a larger proton range pulling-back is needed; correspondingly, a larger minimal MU/spot is desired for plan optimization to maintain the FLASH dose rate for irradiation to OARs.

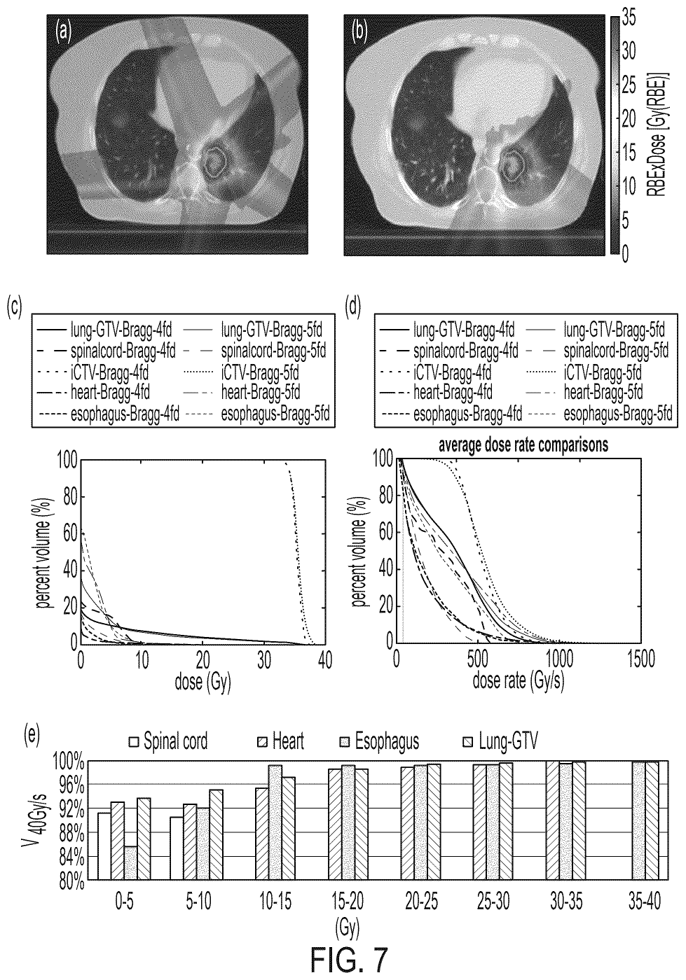

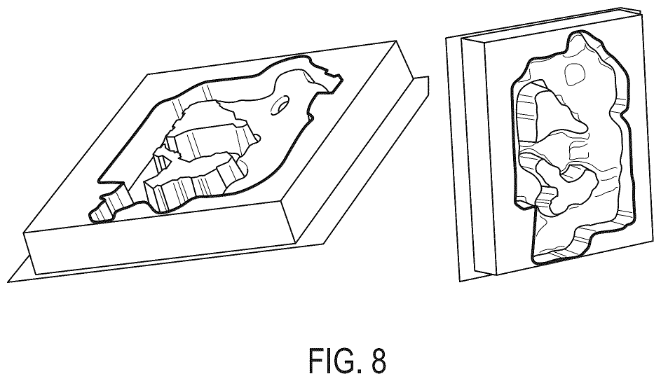

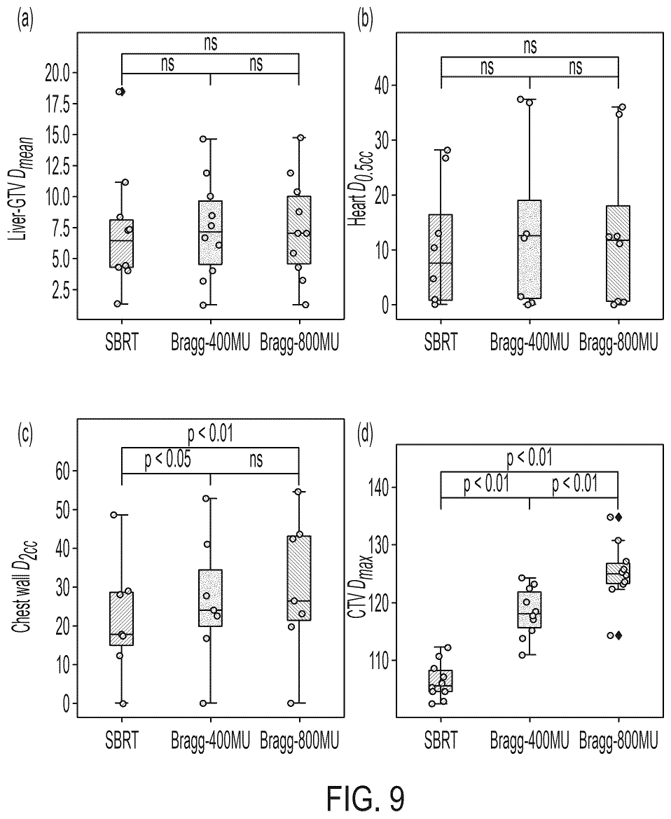

Plan optimization involving multiple parameters, including more freedom will definitely increase the flexibility and possibility for achieving better plan quality. The variable minimal MU/spot, number of beams, and beam angles are key parameters that should generally be considered during treatment planning optimization. As shown in FIG. 7, (a) uses a 4-posterior-oblique-beam arrangement which is close to conventional beam angles compared to that in (b); The contralateral lung is completely spared with almost zero doses, and dose to the esophagus and heart are also significantly reduced (DVHs from FIG. 7(c)); the V_{40Gy/s} (FIG. 7(d)) coverage for the CTV and OARs are similar. This case comparison indicates that the Bragg peak plan has much potential to further reduce OAR doses by optimizing the number of fields and beam angles. Plan robustness is extremely important to achieve the clinically intended dose to the target and OARs. A smearing method is used to consider the range uncertainties from setup and CT HU to stopping power conversion uncertainties. Motion uncertainties can also have an impact on the target treatment. As each field is delivered in under 1 second, the dose interplay effect may not be a concern for each field, while in between fields and fractions, the target motion still needs to be addressed. A 4DCT will be needed to contour moving targets considering the motion amplitude and motion region. Meanwhile, an average image may be helpful to make the treatment plan more robust. Motion mitigation strategies (DIBH, gating, etc.) may also be necessary to reduce the target margin and achieve robust treatment.

The V_{40Gy/s} coverage was slightly worse in Bragg peak plans even when the minimal MU/spot was boosted to 1200 MU/spot. We have analyzed where the low dose rate region is. We define eight dose regions with 5 Gy dose intervals from 0 to 40 Gy to quantitatively assess the dose rate statistical distribution vs. OAR doses. As shown in FIG. 7(e), there are eight dose regions from 0-5 Gy to 35-40 Gy. Most of the volume receiving FLASH dose rate coverage less than 96% had doses lower than 15 Gy. When the OARs doses are higher, the dose rate increases correspondingly. Studies indicated that the FLASH sparing effect might be correlated with the combination of dose, dose rate, and total delivery time. As studies indicated that the FLASH effect might be dose-dependent, we presume this low dose region with a slight loss of V_{40Gy/s} coverage might not be critical or of clinical significance.

FIG. 7(a-d) An example illustrating beam angle optimization. FIG. 7(a) and FIG. 7(b) are the 2D dose distribution using different fields and field angles, FIG. 7(c) and FIG. 7(d) are the DVH, and DRVH comparison, (e) is the V_{40Gy/s} dose rate coverage vs. OAR doses from the low to high dose regions. The left side lung and most of the heart are completely spared using a beam arrangement shown by (b).

### Conclusion

The dedicated designed URS and RS accomplished the mission of pulling proton range back from the target from distally to proximally. This novel method does not require any significant cyclotron/synchrotron or beamline updates to meet the FLASH-RT dose rate threshold using Bragg peaks. The URS is an effective tool to pull back the proton range. With the help of the RC, the proton range can be further tailored to adapt to the distal target contour to achieve a conformal dose distribution. The exit dose can be eliminated with Bragg peak FLASH plans, allowing much better OAR sparing than transmission FLASH plans. An effective inverse optimizer by taking full advantage of planning freedom, e.g., beams, angles, spot maps, spot weightings, etc., is another critical factor to achieve high-quality IMPT plans with a sufficient 3D dose rate for FLASH-RT. An efficient dose rate optimization algorithm and an accurate dose calculation engine are crucial to make Bragg peak FLASH-RT feasible in clinical practice. We have demonstrated an initial cohort planning analysis that demonstrates similar target coverage and uniformity can be maintained using Bragg peaks but with substantially improved OAR sparing relative to transmission plans. This first proof-of-concept study has demonstrated the novel method of combining range pulling back and powerful inverse optimization to achieve FLASH dose rate using currently available machine parameters.

### Example 2

### Treatment of Lung Tumor

A 26-year old male is diagnosed with a lung tumor. The cancerous tissue is imaged and mapped, and a treatment plan is devised. Using an inverse-planning optimization protocol, an optimal URS plate combination was established with the following parameters to coincide the range of the shifted and compensated ionizing radiation with the distal end of the target tissue:

Plate thickness(cm)

| Pull-back (cm) | 14 | 7 | 7 | 3 | 2 | 1 |
|---|---|---|---|---|---|---|
| 28 | 1 | 1 | 1 | 0 | 0 | 0 |
| 26 | 1 | 1 | 0 | 1 | 1 | 0 |
| 27 | 1 | 1 | 0 | 1 | 1 | 1 |
| 29 | 1 | 1 | 1 | 0 | 0 | 1 |
| 30 | 1 | 1 | 1 | 0 | 1 | 0 |

FLASH-RT treatment is then provided to the cancerous tissue in 5 fields spaced at an angle of 72°.

### Example 3

### Treatment of Liver Tumor

A 46-year old female is diagnosed with a liver tumor. The cancerous tissue is imaged and mapped, and a treatment plan is devised. Using an inverse-planning optimization protocol, an optimal URS plate combination was established with the following parameters to coincide the Bragg peak of the shifted and compensated ionizing radiation with the distal end of the target tissue:

Plate thickness(cm)

| Pull-back (cm) | 14 | 7 | 7 | 3 | 2 | 1 |
|---|---|---|---|---|---|---|
| 18 | 1 | 0 | 0 | 1 | 0 | 1 |
| 23 | 1 | 1 | 0 | 0 | 1 | 0 |
| 19 | 1 | 0 | 0 | 1 | 1 | 0 |
| 20 | 1 | 0 | 0 | 1 | 1 | 1 |
| 21 | 1 | 1 | 0 | 0 | 0 | 0 |
| 22 | 1 | 1 | 0 | 0 | 0 | 1 |

FLASH-RT treatment is then provided to the cancerous tissue in 6 fields spaced at an angle of 60°.

### Example 4

### Treatment of Brain Tumor

A 41-year old male is diagnosed with a brain tumor. The cancerous tissue is imaged and mapped, and a treatment plan is devised. Using an inverse-planning optimization protocol, an optimal URS plate combination was established with the following parameters to coincide the Bragg peak of the shifted and compensated ionizing radiation with the distal end of the target tissue:

Plate thickness(cm)

| Pull-back (cm) | 14 | 7 | 7 | 3 | 2 | 1 |
|---|---|---|---|---|---|---|
| 21 | 1 | 1 | 0 | 0 | 0 | 0 |
| 22 | 1 | 1 | 0 | 0 | 0 | 1 |
| 23 | 1 | 1 | 0 | 0 | 1 | 0 |
| 21 | 1 | 1 | 0 | 0 | 0 | 0 |

FLASH-RT treatment is then provided to the cancerous tissue in 4 fields spaced at an angle of 90°.

### Example 5

### Treatment of Brain Tumor

A 21-year old male is diagnosed with a brain tumor. The cancerous tissue is imaged and mapped, and a treatment plan is devised. Using an inverse-planning optimization protocol, an optimal URS plate combination was established with the following parameters to coincide the Bragg peak of the shifted and compensated ionizing radiation with the distal end of the target tissue:

Plate thickness(cm)

| Pull-back (cm) | 14 | 7 | 7 | 3 | 2 | 1 |
|---|---|---|---|---|---|---|
| 26 | 1 | 1 | 0 | 1 | 1 | 0 |
| 32 | 1 | 1 | 1 | 1 | 0 | 1 |
| 27 | 1 | 1 | 0 | 1 | 1 | 1 |
| 28 | 1 | 1 | 1 | 0 | 0 | 0 |
| 33 | 1 | 1 | 1 | 1 | 1 | 0 |
| 29 | 1 | 1 | 1 | 0 | 0 | 1 |
| 30 | 1 | 1 | 1 | 0 | 1 | 0 |
| 31 | 1 | 1 | 1 | 1 | 0 | 0 |

FLASH-RT treatment is then provided to the cancerous tissue in 8 fields spaced at an angle of 45°.

### Example 6

### Treatment of Liver Tumor

A 56-year old female is diagnosed with a liver tumor. The cancerous tissue is imaged and mapped, and a treatment plan is devised. Using an inverse-planning optimization protocol, an optimal URS plate combination was established with the following parameters to coincide the Bragg peak of the shifted and compensated ionizing radiation with the distal end of the target tissue:

Plate thickness(cm)

| Pull-back (cm) | 14 | 7 | 7 | 3 | 2 | 1 |
|---|---|---|---|---|---|---|
| 28 | 1 | 1 | 1 | 0 | 0 | 0 |
| 26 | 1 | 1 | 0 | 1 | 1 | 0 |
| 27 | 1 | 1 | 0 | 1 | 1 | 1 |

FLASH-RT treatment is then provided to the cancerous tissue in 3 fields spaced at an angle of 120°.

### Example 7

### Treatment of Esophageal Tumor

A 51-year old male is diagnosed with an esophageal tumor. The cancerous tissue is imaged and mapped, and a treatment plan is devised. Using an inverse-planning optimization protocol, an optimal URS plate combination was established with the following parameters to coincide the Bragg peak of the shifted and compensated ionizing radiation with the distal end of the target tissue:

Plate thickness(cm)

| Pull-back (cm) | 14 | 7 | 7 | 3 | 2 | 1 |
|---|---|---|---|---|---|---|
| 18 | 1 | 0 | 0 | 1 | 0 | 1 |
| 19 | 1 | 0 | 0 | 1 | 1 | 0 |
| 20 | 1 | 0 | 0 | 1 | 1 | 1 |
| 21 | 1 | 1 | 0 | 0 | 0 | 0 |
| 22 | 1 | 1 | 0 | 0 | 0 | 1 |

FLASH-RT treatment is then provided to the cancerous tissue in 5 fields spaced at an angle of 72°.

### Example 8

### Use of a Compact Flash RT System

### Dose and Dose Rate Calculation

We commissioned the highest 250 MeV beam model of the proton system in our research facility using an in-house treatment planning system (TPS) based on the matRad framework for the single-energy BP treatment planning. We first account for the dose rate model based on the relationship between beam current, minimum monitor unit (MU), and minimum spot time (MST). The beam current directly determines the dose rate, i.e., the dose deposited by the fluence of protons per second into the volume of interest. The minimum MU is determined by the production of beam current and MST. For the proton system to work under the FLASH mode, an MST has to be guaranteed for the delivery of the minimum MU required by the proton delivery system. Such MST may be variable for different systems, and 2 ms and 0.5 ms MST are achievable on a commercial proton system for FLASH studies.

Spot scanning speed determined the spot scanning time between spots. In the context of the PBS paradigm, there exist various definitions of dose rate. In this study, we adopted the average dose rate (ADR) definition proposed by Folkerts et al. This particular definition was considered relatively conservative as it takes into account both spot dwelling and slew times during the PBS delivery process.

The inverse planning process for the PBS Bragg peak method involved a three-step optimization approach. First, during the initial phase of inverse planning, no minimum MU constraints were imposed. A full grid of spots was utilized for the different fields in the treatment plan.

Second, to fulfill the minimum MU constraints and accommodate the dose rate requirement for FLASH delivery, we developed a spot map optimization procedure. This procedure effectively combined neighboring small-weight spots into larger-weight ones, ensuring compliance with the minimum MU constraints.

Finally, in the second phase of inverse planning, the same minimum MU constraint was applied accordingly. By employing this three-step optimization process, we aimed to achieve an optimal treatment plan using the PBS Bragg peak method, considering both the dose rate requirements for FLASH delivery and adherence to minimum MU constraints.

### Universal Range Shifter and Range Compensator Design

Proton energy loss can be described by the Bethe-Bloch equation. This equation primarily depends on two factors: the square of the nuclear charge (Z) and the inverse square of the projectile proton velocity (B). During proton beam delivery, each individual proton spot corresponds to a specific water-equivalent thickness (WET) value. These WET values were precomputed from the TPS. Interpolation techniques were applied to determine the WET values for the entire treatment field. This allowed for the computation of the necessary range pullback's WET, which ensures that the proton beams conform to the desired distal exit target. Due to uncertainties in the spot locations relative to the patient's body as seen from the beam eye's view, a smearing method was utilized. This method involves shifting the positions of the spots by ±3 mm to ±5 mm independently perpendicular to the beam directions from their planned positions.

Subsequently, the maximum WET resulting from these perturbations was assigned to the corresponding spot as the final WET value. This approach helped account for any uncertainties in the spot locations and ensures appropriate dosimetric coverage during treatment delivery.

The WET of each pencil beam proton radiographic track was determined using Equation 1. In this equation, rsp(x,y,z) represents the relative stopping power (RSP) of each voxel in the 3D CT images. The integral step in Equation 1 was precisely calculated using a ray-tracing algorithm. Additionally, the range pullback for each pencil beam was computed as Rᵢ, where R_{Eo} represents the range of the highest energy in water. This calculation allowed for adjustments to the range of the pencil beam to achieve the desired treatment depth based on the specific RSP values encountered along the beam path. By utilizing these calculations, the WET and range pullback values for each pencil beam were determined, enabling accurate treatment planning and delivery based on the patient's individualied anatomical characteristics.

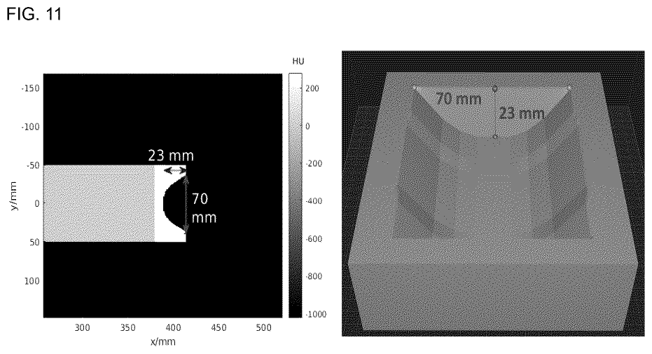

Using the in-house TPS, a pseudo-CT image was computed that includes the URS and RC components (refer to FIG. 11(a)). Additionally, based on the treatment plans, a 3D model (FIG. 11(b)) is generated suitable for 3D printing. The geometry size displayed on the planned range compensator in the CT image perfectly matched the dimensions of the corresponding region in the 3D model. In this case, both measurements were precisely 70 mm, indicating an accurate reproduction of the range compensator according to the planned specifications. This successful alignment between the CT image and the 3D model confirmed the reliability of the 3D printing process and ensures that the range compensator would be accurately replicated for use in treatment delivery.

### Experimental Validation of Bragg Peak FLASH RT

### End-to-End Flowchart

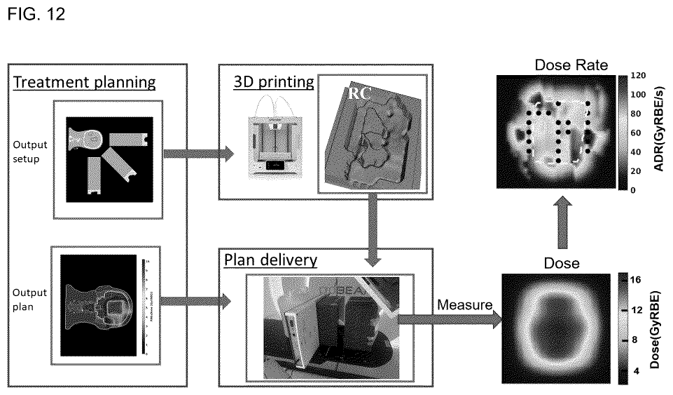

FIG. 12 illustrates the comprehensive end-to-end flowchart, showcasing the design and optimization of Bragg peak plans using the in-house TPS. The dose distribution was calculated employing both proton convolution superposition (PCS) and Monte Carlo (MC) algorithms, ensuring accurate and precise results. 3D printing technology was utilized to generate RCs for each beam. These RCs were then securely affixed to solid water slabs, serving as URS, with their centers aligned to the proton beam radiation center. This setup allowed for precise positioning and alignment during treatment. A 5-cm air gap was designed between the phantom (patient) and URS for sufficient clearance. The Matrix PT was placed behind the phantom at the designed depths to measure the dose compared with the TPS planned dose distribution.

To assess the accuracy of the dosimetry, a gamma analysis was conducted using a 3 mm/3% criterion and a 10% dose cutoff. However, it is important to note that the Matrixx PT, the device used for this validation, experiences ion recombination when subjected to a FLASH dose rate. To overcome this challenge and validate the feasibility of the Bragg peak FLASH method, we delivered the treatment using a clinical beam current of 2 nA. By utilizing this reduced beam current, ion recombination effects were avoided and reliable dosimetry measurements during validation ensured.

The FLASH dose rate was further characterized using a hybrid method, combining the measured time structure of delivered spots with the measured dose to reconstruct the 3D dose rate distribution. A previous study has confirmed the suitability of a high temporal resolution 2D strip-segmented ionization chamber array (SICA) for monitoring the transmission FLASH beam. In this case, the SICA with a sampling rate of 20 KHz (50 μs per event) can accurately measure the time structure, enabling the reconstruction of the 3D dose rate. By utilizing this hybrid method, we could characterize and understand the intricate dose rate patterns associated with the FLASH technique.

### Demonstration of Inhomogeneous Phantom

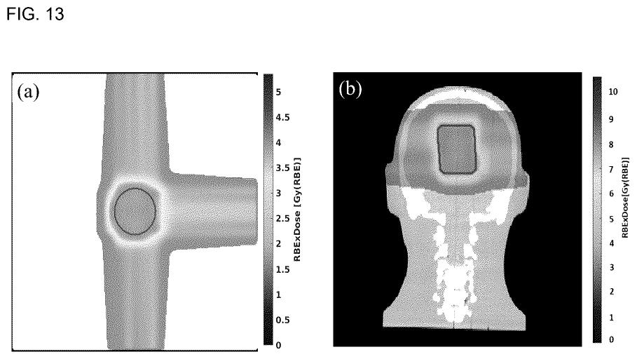

A homogeneous solid water phantom with a cylindrical target (6 cm in diameter) located at the center of the phantom was used to validate accuracy. A 3-field treatment plan using single-energy of 250 MeV proton beams was optimized to deliver a 5 Gy uniform dose to the target, as depicted in FIG. 13(a). The field angles were 0, 90, and 180 degrees, respectively. A minimum MU constraint of 200, equivalent to a beam current of 85 nA, was used for plan optimization.

Because the optimization process involved multiple fields, the resulting field dose distribution was non-uniform. To evaluate the accuracy, the Matrix PT detector was positioned behind the solid water phantom at three distinct depths: 9 cm, 12 cm, and 15 cm. These measurements were utilized to generate dose maps for subsequent gamma analysis, allowing a comparison with the planned dose maps. Achieving precise RC alignment is crucial for accurately tracking the Bragg peak distal edge. To ensure this alignment, kV imaging alignment was conducted. This process involved identifying and aligning the RC's center with the delivered fields' radiation center. By aligning the RC precisely, we could enhance the accuracy of the treatment and facilitate the desired dose deposition at the intended location.

### Demonstration in Anthropometric Head-Neck Phantom

An anthropometric head-neck phantom was utilized to validate the accuracy of Bragg peak treatment and delivery in heterogeneous tissue. This phantom offered the flexibility of three different depths where films or detector arrays could be inserted for dose measurement purposes, as shown in FIG. 13(b). A three-field arrangement delivered a 10 Gy uniform dose to the brain tumor, and the beam angles were 0, 90, and 270, respectively. In a similar manner, the RCs and URS were specifically designed within our in-house TPS. These components were then fabricated using a 3D printer, ensuring accurate and precise construction. The setup accuracy was ensured by kV imaging, which also includes the isocenter identification and close registration between the RC and head-neck phantom according to the treatment plan. The Matrix PT detector was placed behind the head-neck phantom to measure the dose distribution for comparison.

### Results

### Bragg Peak FLASH Treatment Plans

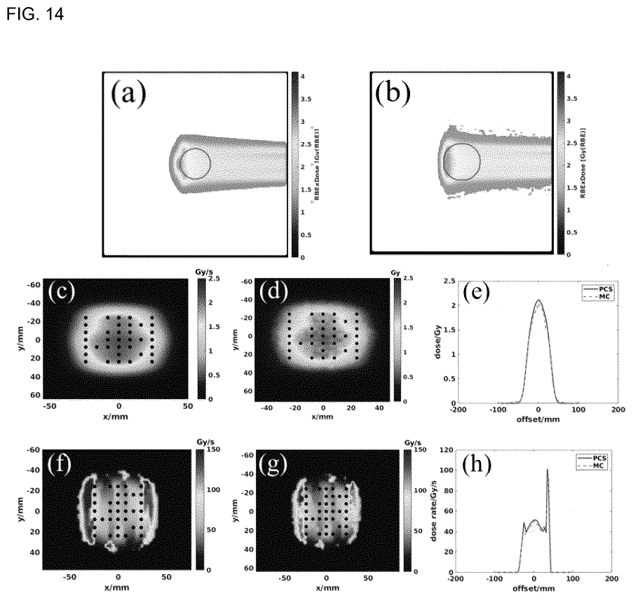

The treatment plans for the homogeneous phantom are depicted in both Figures, showcasing the utilization of both PCS in FIG. 14(a) and the MC algorithm in FIG. 14(b). In FIGS. 14(c-d), the 2D dose distribution is depicted, showcasing the spatial distribution of the planned dose at a representative depth, and the black dots represent the spot placement. The comparison between the PCS and MC plans reveals a high level of agreement, as indicated by the line profiles presented in FIG. 14(e). The dose rate distributions for a minimum of 508 MU/spot, corresponding to 215 nA, were reconstructed using the measured time structures and calculated doses in FIGD. 14(f-g).

It is noticeable that the dose rate distributions between the PCS and MC plans exhibited similar streak-like patterns, as well as “hotter” edges in the dose rate maps. These observations were consistent with previous simulation results. The streak-like patterns in the dose rate distributions are a characteristic feature of PBS proton therapy. This scanning technique involves delivering a sequence of proton spots to cover the treatment area. The streaks in the dose rate maps corresponded to the paths of individual proton spots as they traverse the target region. It is worth noting that the edge portion of the MC dose rate distribution can be smaller than that of the PCS plan. The slight difference in dose rate between MC and PCS calculations can be attributed to the inherent variations in the modeling and calculation approaches employed by each algorithm. As a highly detailed simulation method, the MC algorithm accounts for a wider range of physical interactions and secondary particle production, which have better accuracy in scattered dose estimation and can affect the dose rate distribution. The MC calculation gives a slightly lower dose in the high-dose region, which can contribute to the observed reduction in the edge portion of the dose rate distribution compared to the PCS plan.

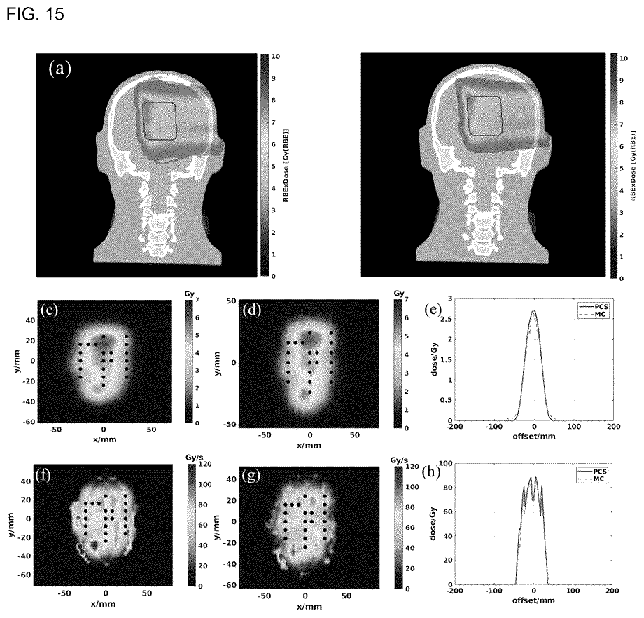

In the head-neck treatment planning, both the PCS and MC dose calculations demonstrated good agreement, as evidenced by the line profiles obtained from the dose maps presented in FIG. 15 . FIGS. 15(f-h) depicts the calculated 2D dose rate distributions. Consistently with previous observations in a uniform solid water phantom, the dose rate obtained using the MC algorithm was slightly lower compared to the results from the PCS algorithm. This finding is also reflected in the head-neck treatment planning scenario. The FLASH coverage V40Gy/s, representing the volume receiving a dose at a rate of 40 Gy/s, was 96.1% for the target and 50.4% for the brain tissue when using PCS, and 95.3% and 36.6%, respectively, when using MC. Both dose calculations for the phantom study showed that the PCS and MC can maintain similar results with a dose calculation difference of less than 1%. The lower dose rate coverage in brain tissue corresponds to the lower dose regions, i.e., proximal and lateral regions of the Bragg-peak beams. MC's calculated dose rate is even lower due to the larger low dose spread modeled, which was not modeled in PCS.

When comparing the dose rate distributions between MC and PCS, it is common to observe that the MC algorithm yields lower dose rates due to the inclusion of a larger low-dose spread. This phenomenon is a result of the MC algorithm's enhanced accuracy in modeling scattered doses and its broader consideration of physical interactions.

### Measurement Results

### Use Homogeneous Solid Water Phantom

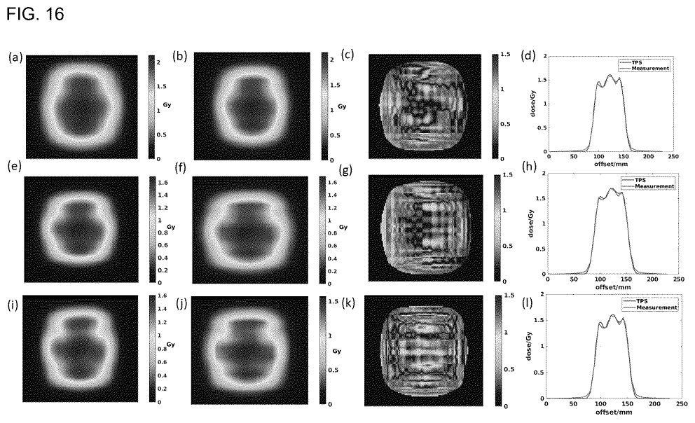

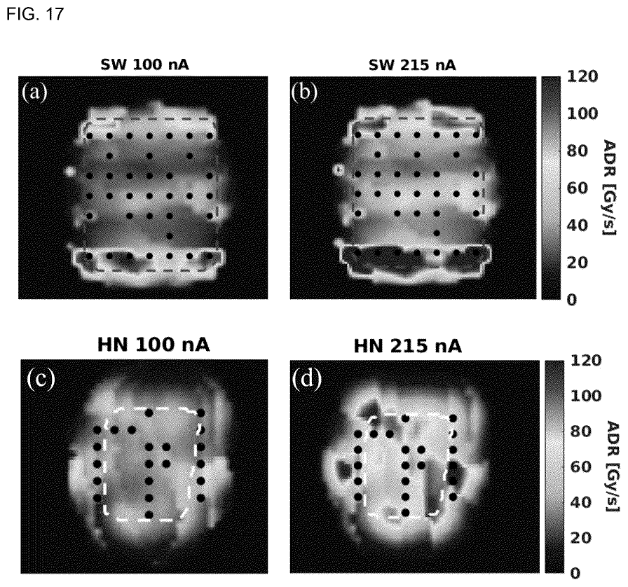

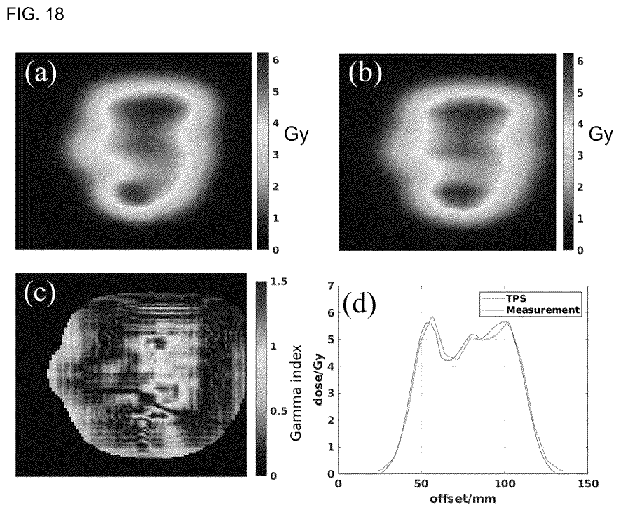

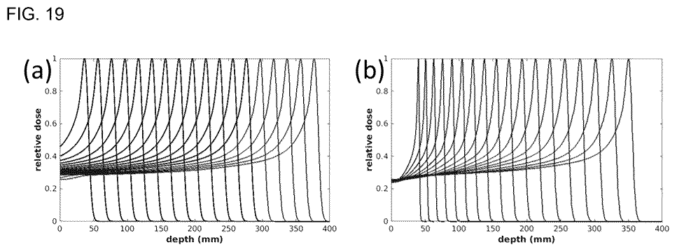

The Matrix PT was placed at 3 different depths of 15, 12, and 9 cm of the solid water phantom. FIG. 16 shows the dose comparison between TPS calculation and measurement for the 3 depths, where the gamma analysis results are also included. A 3 mm/3% criterion and 10% cutoff threshold were applied for the gamma analysis. The 2 D plane dose gamma passing rate were 99.9%, 99.2%, 99.7% for the 3 depths, respectively. The dose rate distribution was reconstructed, as shown in FIG. 17. The dose rate coverage for the targets were 39.5% and 85.6% under 2 different nozzle beam currents of 100 and 215 nA, respectively.

### Head-Neck Phantom

In the head-neck phantom measurement, we also noticed very close results for the dose maps between the plan and measurement, with a gamma analysis passing rate of 97.05%, a 3 mm/3% criterion, and a 10% cutoff threshold. The dose rate distribution was reconstructed, as shown in FIG. 17 (c) and (d). The dose rate coverage for the targets were 26.4% and 100% for 100 and 215 nA, respectively.

### Discussion

We successfully developed the pristine Bragg peak technique, covering the entire process from treatment planning to actual delivery, by using single-energy Bragg peak proton beams. This achievement was made possible through the combined use of URS and 3D-printed RCs, both supported by our in-house developed TPS that utilizes PCS and MC dose calculation algorithms. This method provides a new solution for implementing single-energy PBS IMPT. It eliminates the need for costly energy degradation selection systems, significantly simplifying proton beamlines and leading to considerable cost savings. Additionally, it reduces the need for extra downstream components used in shaping the beam profiles. This transforms the field by offering an efficient and cost-effective option for delivering accurate proton therapy treatments.

Experimental validation was a crucial step in demonstrating the feasibility of this economical solution for delivering conformal FLASH RT. This study verified the dosimetry accuracy between planning and delivery. The design and manufacture of the 3D-printed RCs were verified by matching the calculation and measurement. As MC is a more accurate algorythm, the physical range pullbacks, air gaps, and tissue boundaries can be well modeled in MC, offering a useful benchmark for the PCS method. Our result has shown a very close match between the PCS and MC in both solid water and head-neck phantoms.

The 3D-printed RCs have to exactly match the design to achieve the Bragg peak distal tracking, and our result indicates a close match. 3D printing is only used for a very narrow piece (depth<5 cm) of the RC in a couple of centimeters with the URS solid water slabs. 3D printing technology has become more available in radiation therapy for different purposes. As the thickness of the RC can be precisely controlled, the speed for 3D printing will not be an issue for efficient clinical use. Matrix PT measured the delivered doses at designed depths. The planned and delivered dose maps agree well with our study with very high gamma passing rates for multiple depths of measurement, which confirm the accuracy of the design and manufacture of the 3D printed RC for Bragg peak distal tracking.

The single-energy Bragg peak method applies appropriately to the FLASH delivery, as only one energy is used without the time-consuming energy switching time of previous systems, and as there can be a very rapid delivery of the field given sufficiently high minimum MU and low MST that corresponds to very high beam current.

In our work, the dose rate was calculated on realistic values provided by vendors and previous literature. The key parts of average dose rate calculation are the spot dwelling time and spot scanning speed, and they can be reliably modeled given the correct machine parameters. The average dose rate, though a relatively conservative dose rate calculation method, can be sensitive to the specific dose threshold chosen in determining the dose accumulation time windows, which can also be affected by different beam models, as seen in the PCS and MC results in our study. The tails due to the MC beam model may cause the starting and ending points to be wider than that of PCS, therefore ending up with smaller ADR values.

This work successfully demonstrated the feasibility of the novel methodology using single energy for Bragg peak FLASH delivery. The novel Bragg peak tacking technique could be applied based on realistic proton machine parameters. The planned dosimetric and dose rate distribution matched the measurement well. A single energy-layer proton beam from a cyclotron for conformal Bragg peak FLASH RT can potentially be a promising solution for preclinical and clinical cancer treatment applications.

### Example 9

### Treatment of Liver Tumor

A 43-year old female is diagnosed with a liver tumor. The cancerous tissue is imaged and mapped, and a treatment plan is devised. Using an inverse-planning optimization protocol, an optimal URS plate combination was established with the following parameters to coincide the Bragg peak of the shifted and compensated single energy ionizing radiation with the distal end of the target tissue:

Plate thickness(cm)

| Pull-back (cm) | 14 | 7 | 7 | 3 | 2 | 1 |
|---|---|---|---|---|---|---|
| 18 | 1 | 0 | 0 | 1 | 0 | 1 |
| 23 | 1 | 1 | 0 | 0 | 1 | 0 |
| 19 | 1 | 0 | 0 | 1 | 1 | 0 |
| 20 | 1 | 0 | 0 | 1 | 1 | 1 |
| 21 | 1 | 1 | 0 | 0 | 0 | 0 |
| 22 | 1 | 1 | 0 | 0 | 0 | 1 |

FLASH-RT treatment without the use of an energy selection system is then provided to the cancerous tissue in 6 fields spaced at an angle of 60°.

### Example 10

### Treatment of Brain Tumor

A 47-year old male is diagnosed with a brain tumor. The cancerous tissue is imaged and mapped, and a treatment plan is devised. Using an inverse-planning optimization protocol, an optimal URS plate combination was established with the following parameters to coincide the Bragg peak of the shifted and compensated single energy ionizing radiation with the distal end of the target tissue:

Plate thickness(cm)

| Pull-back (cm) | 14 | 7 | 7 | 3 | 2 | 1 |
|---|---|---|---|---|---|---|
| 21 | 1 | 1 | 0 | 0 | 0 | 0 |
| 22 | 1 | 1 | 0 | 0 | 0 | 1 |
| 23 | 1 | 1 | 0 | 0 | 1 | 0 |
| 21 | 1 | 1 | 0 | 0 | 0 | 0 |

FLASH-RT treatment without the use of an energy selection system is then provided to the cancerous tissue in 4 fields spaced at an angle of 90°.

### Example 11

### Treatment of Brain Tumor

A 31-year old male is diagnosed with a brain tumor. The cancerous tissue is imaged and mapped, and a treatment plan is devised. Using an inverse-planning optimization protocol, an optimal URS plate combination was established with the following parameters to coincide the Bragg peak of the shifted and compensated single energy ionizing radiation with the distal end of the target tissue:

Plate thickness(cm)

| Pull-back (cm) | 14 | 7 | 7 | 3 | 2 | 1 |
|---|---|---|---|---|---|---|
| 26 | 1 | 1 | 0 | 1 | 1 | 0 |
| 32 | 1 | 1 | 1 | 1 | 0 | 1 |
| 27 | 1 | 1 | 0 | 1 | 1 | 1 |
| 28 | 1 | 1 | 1 | 0 | 0 | 0 |
| 33 | 1 | 1 | 1 | 1 | 1 | 0 |
| 29 | 1 | 1 | 1 | 0 | 0 | 1 |
| 30 | 1 | 1 | 1 | 0 | 1 | 0 |
| 31 | 1 | 1 | 1 | 1 | 0 | 0 |

FLASH-RT treatment without the use of an energy selection system is then provided to the cancerous tissue in 8 fields spaced at an angle of 45°.

### Example 12

### Treatment of Liver Tumor

A 49-year old female is diagnosed with a liver tumor. The cancerous tissue is imaged and mapped, and a treatment plan is devised. Using an inverse-planning optimization protocol, an optimal URS plate combination was established with the following parameters to coincide the Bragg peak of the shifted and compensated single energy proton beam with the distal end of the target tissue:

Plate thickness(cm)

| Pull-back (cm) | 14 | 7 | 7 | 3 | 2 | 1 |
|---|---|---|---|---|---|---|
| 28 | 1 | 1 | 1 | 0 | 0 | 0 |
| 26 | 1 | 1 | 0 | 1 | 1 | 0 |
| 27 | 1 | 1 | 0 | 1 | 1 | 1 |

FLASH-RT treatment without the use of an energy selection system is then provided to the cancerous tissue in 3 fields spaced at an angle of 120°.

### Example 13

### Treatment of Esophageal Tumor

A 50-year old male is diagnosed with an esophageal tumor. The cancerous tissue is imaged and mapped, and a treatment plan is devised. Using an inverse-planning optimization protocol, an optimal URS plate combination was established with the following parameters to coincide the Bragg peak of the shifted and compensated single energy proton beam with the distal end of the target tissue:

Plate thickness(cm)

| Pull-back (cm) | 14 | 7 | 7 | 3 | 2 | 1 |
|---|---|---|---|---|---|---|
| 18 | 1 | 0 | 0 | 1 | 0 | 1 |
| 19 | 1 | 0 | 0 | 1 | 1 | 0 |
| 20 | 1 | 0 | 0 | 1 | 1 | 1 |
| 21 | 1 | 1 | 0 | 0 | 0 | 0 |
| 22 | 1 | 1 | 0 | 0 | 0 | 1 |

FLASH-RT treatment without the use of an energy selection system is then provided to the cancerous tissue in 5 fields spaced at an angle of 72°.

### Example 14

### Proton Arc Therapy

The current approach to PAT involves using either multiple energy layers per sub-arc or a mono-energy per sub-arc (ARC), depending on the energy switching capabilities of cyclotron or synchrotron accelerators. This method often restricts the efficiency of treatment delivery. We propose a novel technique called quasi-distal tracking, which utilizes the highest energy layer and universal range shifter (URS) to facilitate rapid spot arc therapy (RAPIDSPARC™).

### Methods:

Utilizing a single-energy layer, which features the highest beam current, not only preserved ultra-fast delivery but also eliminated the extended energy switching time exceeding one second. A URS composed of variable thickness plates effectively adjusted the range of the single-energy beam for each sub-arc, facilitating rapid delivery. By incorporating a Genetic Algorithm (GA) to optimize the sequence of range layers for each sub-arc, along with greedy spot map optimization and Linear Energy Transfer (LET) optimization, we can achieve plan quality comparable to multiple-energy layer approaches while enhancing LET for critical Organs at Risk (OARs). This novel RAPIDSPARC™ was been evaluated using a C-shaped phantom target and clinical case studies. The plan quality has been benchmarked against standard Intensity-Modulated Proton Radiotherapy (IMPT) and ARC, demonstrating its efficacy.

### Results:

The GA and greedy spot map optimization effectively achieved plan quality for RSARC. The LET optimization significantly reduces the high LET to critical OARs. The delivery time of the RAPIDSPARC™ was reduced by ˜77% compared to ARC, but with comparable dosimetry characteristics.

### Conclusion:

The innovative RAPIDSPARC™ technique achieves plan quality comparable to ARC, yet surpasses that of IMPT, showcasing its significant potential for rapid proton arc delivery in clinical settings.

### Innovation:

Conventional methods of proton arc therapy employ either multiple energy layers or a single energy layer per sub-arc (as described herein), determined by the energy-switching capabilities of cyclotron or synchrotron accelerators. This approach frequently hampers the efficiency of therapy delivery. Our proposed novel technique, termed quasi-distal tracking, leverages the highest energy layer in conjunction with a universal range shifter (URS) to expedite and enhance the efficacy of proton arc therapy, referred to as RAPIDSPARC™. This method enhances delivery efficiency and ensures robust performance by utilizing the single energy, which features a broader Bragg peak spanning from the proximal 80% to the distal 80%. This characteristic helps maintain consistent and reliable delivery.

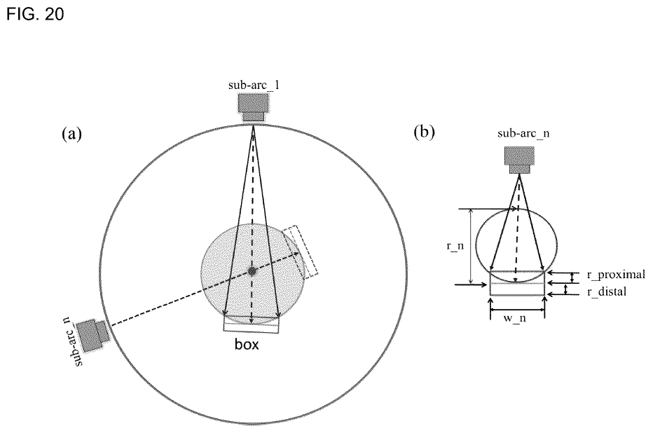

Key results: The Genetic Algorithm (GA) and greedy spot map optimization significantly improve the plan quality of RAPIDSPARC™. As shown in FIG. 20, RAPIDSPARC™ achieves a delivery time that is about 77% shorter than that of ARC, yet it retains comparable dosimetry characteristics

### Introduction

Proton therapy has emerged as a revolutionary cancer treatment modality offering superior dose conformity and reduced radiation exposure to healthy tissues compared to conventional photon therapy. However, traditional IMPT often involved fixed beam angles, limiting its ability to conform to complex tumor shapes. This is where proton arc therapy comes in, offering a significant advancement in the field.

Inspired by volumetric modulated arc therapy (VMAT) in photon therapy, proton arc therapy utilizes a continuous beam rotation around the patient, allowing for improved conformity to complex tumor geometries. While offering enhanced plan quality through increased beam angles and multiple energy layers, one key challenge remains-treatment delivery time. Lengthy treatment times hinder the broader clinical adoption of proton arc therapy. Researchers have actively addressed this issue, proposing an optimization method that reduces the number of energy layers and optimizes their sequencing, leading to shorter treatment times.

While proton arc therapy offers enhanced dose conformity compared to intensity-modulated proton therapy (IMPT), this benefit can come at the cost of increased low-dose/low-linear energy transfer (LET) volumes. To address this trade-off and improve normal tissue sparing, researchers proposed a novel approach utilizing LET and relative biological effectiveness (RBE) optimization. This technique aims to simultaneously increase LET and RBE within the target volume while decreasing these values in healthy tissues, ultimately enhancing tumor control without compromising target coverage. Furthermore, researchers proposed an energy layer selection method that can assign a single energy layer per discretized direction.

An alternative strategy focuses on lowering the LET delivered to organs at risk (OARs). This method involves strategically placing proton spots within the center of the target volume, effectively shielding surrounding tissues. Additionally, this approach utilizes monoenergetic beams, which can offer a balance between treatment efficiency and plan quality. The primary culprit behind these extended delivery times is the energy selection system's slow switching mechanism.

Existing cyclotron-based systems rely on a degrader after proton extraction from the accelerator, requiring adjustments to magnetic fields for each energy change. This process can be time-consuming, with energy layer switch time on the order of 200 milliseconds (ms) in a cyclotron-based system. energy layer switch time is even longer in the synchrotron-based system, exceeding 1 second. In order to achieve ultra-high dose rate delivery using Bragg peak for treatment, researchers proposed a novel proton range modulation method. By using a binary range shifter, the energy layer switch time can be shortened to less than a few ms. Using a similar technique to replace the degrader in proton ARC beam delivery significantly reduces the energy layer switching time. This method also improves beam transport efficiency due to the removal of the energy selection system. Consequently, a very high beam current can be preserved, and a higher Minimum Monitor Unit (MMU) per spot can be used in planning and delivery, further shortening beam delivery time and even reaching an ultra-high FLASH dose rate for conventional fractionation treatment.

This study introduces a quasi-distal tracking proton ARC therapy, a novel approach for rapid spot arc delivery (RAPIDSPARC™). RAPIDSPARC™ utilizes the highest energy of cyclotron or single energy from synchrotron via a universal range shifter (URS) for range adjustment to eliminate frequent energy layer switching, which can significantly reduce treatment times. The delivery from multiple sub-arcs always places the Bragg peak inside the target, facilitating the high LET limited to the target. Thus, it can effectively preserve high LET regions to target and better protect the OARs in the beam path. Additionally, during our RAPIDSPARC™ optimization, the LET objective functions are incorporated to optimize the biological effectiveness within the target while maintaining superior dosimetric outcomes.

### Methods and Materials

### The Physics Characteristics of Mono-Energy Arc and RAPIDSPARC™

RAPIDSPARC™ uses URS to adjust the proton range for each beam angle. Due to the stochastic energy loss from inelastic Coulomb interactions between protons and URS, energy “straggling” leads to a widened energy spectrum for each intended lower energy beam. As illustrated in FIG. 19(a; left image), the ProBeam 250 MeV beam was pulled back every 2 cm in water. While compared to a mono-energetic proton beam, energy degradation, and selection systems are used to purify the energy spectrum, which usually has a much narrower Bragg peak for lower depth, as shown in FIG. 19(b; right image) the conventional Bragg peaks at different energies in water.

In the disclosed URS PAT treatment method, the initial energy remains constant; therefore, all the Bragg peaks preserve a constant shape, no matter their depths in tissue. The conventional mono-energetic proton beam has a much narrower peak for lower energy and shallow depth due to the less range straggling and narrow initial energy spectrum.

### A Quasi-Distal Tracking using a Single-Energy Layer

This study used a proton beam with the single energy, from a cyclotron or synchrotron-based system. The quasi-distal tracking method was introduced to achieve uniform dose coverage in the target region. At each beam angle, only one range pull was used. As shown in FIG. 20, for each beam angle, ray tracing is used to compute the water equivalent depth of the voxel in the target. Based on this information, the Bragg peak is placed distally inside the target, equivalent to a single-energy layer for each sub-arc, and the spots will cover the box area shown in FIG. 20(a).

Considering the target geometry's complexity, each sub-arc's range pull-back needs to be further optimized. A combinatorial optimization algorithm can determine the optimal range of each sub-arc (r_n, as shown in FIG. 20(b)). We used a GA (genetic algorithm) to solve the range selection/pull-back. The range on one beam angle was indexed from 1 to N; each indexed range layer can be taken as a gene in GA. All the range sequences in the arc plan generate a chromosome, and by crossover between chromosomes and mutations in the chromosome, we randomly search for the optimal result.

A RAPIDSPARC™ optimization can start from an objective function F, as shown in equation 1. The dose, LET, and MMU were optimized simultaneously.

$$F = {f}_{D} + {f}_{W} + {f}_{LET} \qquad ( eq . 6 )$$

f_{d} represents the dose constraints for OARs and target metrics used in conventional PBS or Intensity-Modulated Proton Therapy (IMPT) optimization. f_{w} regulates the MMU constraints to achieve a sparse spot distribution for each sub-arc. f_{w} applies a soft MU boundary, pushing the MU per spot to be as high as possible.

$${f}_{W} ( x ) = \{ \begin{matrix}\frac{x ( {x}_{min} - x )}{{x}_{min}^{2}} , & if x < {x}_{min} \\ 0 , & otherwise\end{matrix} \qquad ( eq . 7 )$$

If x<xₘᵢₙ/2, f_{w} pushes the x towards 0, If xₘᵢₙ<x<xₘᵢₙ, f_{w} pushes the x towards to xₘᵢₙ.

In order to reduce high LET dose in critical OARs, we induce the over LET objective function U.

$${U}_{k} ( d , l ) = \frac{1}{{N}_{k}} {\sum }_{i = 1}^{{N}_{k}} H ( {d}_{i} - {d}_{o}^{k} ) H ( {l}_{i} - {l}_{0}^{k} ) {( {l}_{i} - {l}_{0}^{k} )}^{2} \qquad ( eq . 8 )$$

Here, Nₖ is the total number of voxels of the k-th ROI, i is the index of the voxel in ROI, dᵢ is the dose of the i-th voxel, and d₀ is the dose threshold to exclude the low dose voxel. I₀ is the LET threshold. H(x) is a piecewise function, where if variable x<0, H(x)=0. Otherwise, the value of H(x) is 1.

Similarly, under the LET objective function L regulates the LET distribution to the target, where desire is as high as possible, as shown in equation 9.

$${L}_{k} ( d , l ) = \frac{1}{{N}_{k}} {\sum }_{i = 1}^{{N}_{k}} H ( {d}_{i} - {d}_{o}^{k} ) H ( {l}_{0}^{k} - {l}_{i} ) {( {l}_{i} - {l}_{0}^{k} )}^{2} \qquad ( eq . 9 )$$

### Delivery Time Modeling

An empirical time estimation model was implemented for the mono-energy arc, which was clinically used to predict the delivery time for real patient treatment based on ProBeam clinical parameters, and its accuracy has been validated. The RAPIDSPARC™ model was based on the MMU and spot time, which is consistent with the FLASH delivery model. The delivery time estimation method can be found in a study by Kang et al. 2021. For a ProBeam, the gantry completes one full rotation in 60 seconds. Two milliseconds (ms) to deliver an MMU per spot was used, and the spot scanning speed was 10 mm/ms. The energy layer switch time for ProBeam system and RAPIDSPARC™ were set to be 1000 ms and 50 ms, respectively.

The total treatment time, as defined in Equation 10, comprises three components: the interval time between two gantry angles, the beam delivery time and the proton scanning time. In Equation 5, the ti represents the interval time between two gantry angles, if the gantry rotation time is longer than the energy layer switch time then, ti is the gantry rotation time otherwise, it equals the energy layer switch time. The second term corresponds to the beam deliver time, for the i-th beam angle, the beam deliver time is the sum of the spot weights wᵢⱼ divided by the beam current li. The third term represents scanning time, for the i-th beam angle, the beam scanning time is the sum of the spots distance divided by scanning speed along x and y direction, respectively.

$$T = ( N - 1 ) * t 1 + {\sum }_{i = 1}^{N} {\sum }_{j = 1}^{{M}_{i}} \frac{{w}_{i j}}{{l}_{i}} + {\sum }_{i = 1}^{N} {\sum }_{j = 1}^{{M}_{i} - 1} \frac{\vert {x}_{ij + 1} - {x}_{ij} \vert}{{v}_{x}} + \frac{\vert {y}_{ij + 1} - {y}_{ij} \vert}{{v}_{y}} \qquad ( eq . 10 )$$

### Dosimetry Studies using Both Phantoms and Patient Cases

To demonstrate the feasibility of RAPIDSPARC™, we applied this technique to a phantom, six prostate, and six complex head and neck patients. Clinical IMPT plans served as the baseline, representing current standard care and were administered to patients. Both mono-energy arc and RAPIDSPARC™ techniques were optimized to quantitatively evaluate plan quality, dose rate, and delivery efficiency. The phantom case utilized a regimen of 2 Gy per fraction over 30 fractions. Prostate treatment employed stereotactic body radiation therapy (SBRT) to deliver 40 Gy in 5 fractions. Head and neck treatments included either simultaneous integrated boost (SIB) or sequential boost techniques. Clinical metrics for prostate and head-neck patients were evaluated across these three methodologies.

### Results

### Dosimetry Performance Among Conventional IMPT. ARC, and RAPIDSPARC™

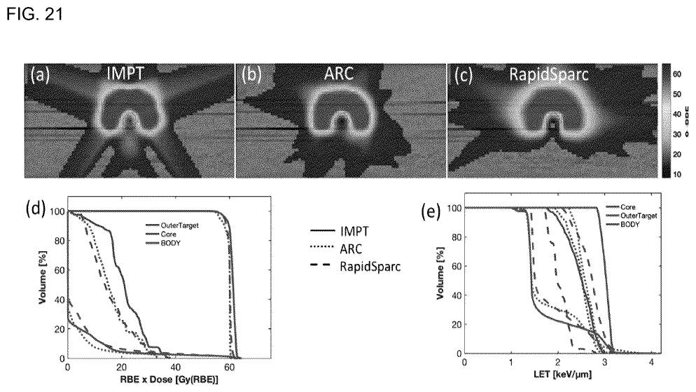

FIG. 21 (a)-(d) show the 2D dose distribution as well as dose-volume histograms for the TG 119 C-shaped target phantom using conventional IMPT, ARC, and RAPIDSPARC™. Both arc methods achieve comparable target dose coverage and lower doses in the OAR region as shown in DVHs in (d). Both ARC plans exhibit high volume for low dose region as indicated by Body DVH as the low dose spread out to more area from the arc therapy. FIG. 21(e) shows the dose averaged LET volume histograms. By applying an upper limit on Core OAR, RAPIDSPARC™ plan reduces high LET volume in the Core region.

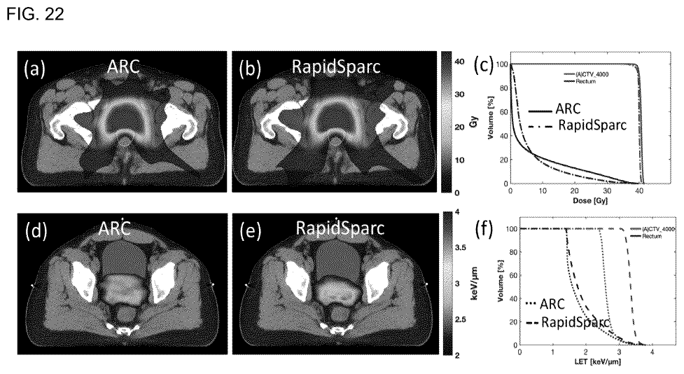

FIG. 22 illustrates the dosimetry and LET comparison for a selected prostate patient. For prostate cases, both ARC and RAPIDSPARC™ therapy plans achieved excellent dose conformity within the target region. However, when examining the critical OAR rectum, the RAPIDSPARC™ plan showed a larger low-dose region compared to the ARC plan. In the high-dose region, the RAPIDSPARC™ plan is more conformal and results in an even lower volume for the high-dose region, as evidenced by the DVHs in FIG. 22(c). The RAPIDSPARC™ plan used a larger spot size than the mono-energetic ARC plan, inherently creating a broader dose distribution.

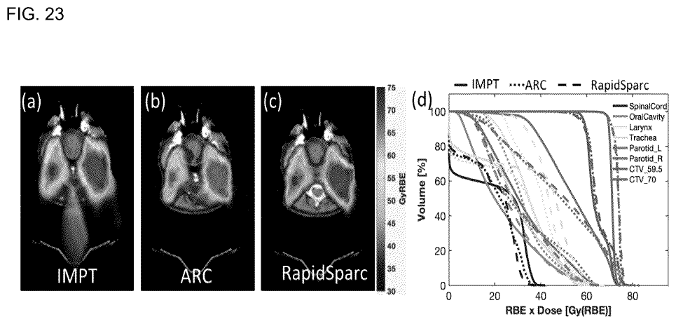

Despite the larger low-dose volume in the rectum, the RAPIDSPARC™ plan has a significant advantage. By applying an upper LET function to the rectum and a lower LET function to the target, the dose-averaged LET in the target region was strategically increased. This could potentially enhance tumor control while maintaining similar rectum sparing levels compared to the ARC plan. In simpler terms, the RAPIDSPARC™ plan achieved comparable protection for healthy organs while potentially improving tumor control. For more complex HN cases, the high-dose region demonstrates superior conformity to the target compared to the IMPT plan, which can be identified from the critical OARs from the 2D and DVH comparison. FIG. 23 demonstrates selected HN cases using SIB techniques, with target dose 59.5Gy and 70Gy in 35 fractions, the fraction dose is 1.7 and 2 Gy, respectively. Particularly, critical structures such as the spinal cord and parotid gland received a lower dose with the ARC plans compared to IMPT. This improved sparing of OARs is attributed to the greater number of beam angles used in arc therapy, leading to improved dose conformity.

Table 4 compares the dosimetric metrics for 1 head and neck (HN) patient who was previously treated using conventional IMPT at our center. All IMPT plans and dose metrics were reviewed and approved by oncologists for clinical delivery, serving as reference baselines compared to the ARC and RAPIDSPARC™ techniques. RAPIDSPARC™ achieved the same target coverage as IMPT and ARC, with dose metrics comparable to or even lower than those of the clinical IMPT and ARC techniques. Notably, for the OARs close to the high-dose region, RAPIDSPARC™ reduced the volume of the high-dose region and achieved a lower maximum dose. The synergy of novel RAPIDSPARC™ and optimization approach resulted in improved conformity. As demonstrated in Table 4, the conformity index of RAPIDSPARC™ is 1.39 compared to 1.41 for the IMPT plans.

TABLE 4
Dosimetric characteristics for 3 techniques adhering to
clinical constraints in 6 head and neck cancer patients.

| ROIs | Metrics | IMPT | ARC | RAPIDSPARC ™ |
|---|---|---|---|---|
| CTV70 | D95 | 99.10% | 100.00% | 100.00% |
| | D99 | 97.00% | 96.00% | 96.80% |
| (A)OralCavity | Dmean | 2358.8 | 3280.4 | 2723.1 |
| BrachialPlexus_{—} | Dmax | 7321 | 4590.8 | 4776.6 |
| L | | | | |
| BrachialPlexus_{—} | Dmax | 6178.7 | 4728.4 | 5045.2 |
| R | | | | |
| BrainStemCore | Dmax | 4250.2 | 4039.5 | 3628.7 |
| BrainStemSurf | Dmax | 4855 | 4473.4 | 4258.9 |
| Chiasm | Dmax | 1466.3 | 132.2 | 1245.1 |
| Cochlea_L | Dmean | 2795.3 | 1178.7 | 2419.5 |
| Cochlea_R | Dmean | 2966.5 | 2607.6 | 2462.9 |
| ConstrMuscle | Dmean | 4611.5 | 4822.4 | 4768.5 |
| Esophagus | Dmean | 1707.3 | 1956.7 | 2027.5 |
| Larynx | Dmean | 3554 | 3994.8 | 4318.2 |
| Lens_L | Dmax | 50.4 | 262.5 | 466.6 |
| Lens_R | Dmax | 126.3 | 309.5 | 631.5 |
| Mandible | Dmax | 7315.8 | 7603.6 | 7108 |
| | D5cc | 5970.4 | 7199.3 | 6919.1 |
| OpticNerve_L | Dmax | 2570.7 | 558.4 | 2133.2 |
| OpticNerve_R | Dmax | 2972.4 | 970.3 | 2660.2 |
| Parotid_L | Dmean | 5392.8 | 4702.5 | 4753.2 |
| Parotid_R | Dmean | 3216 | 3118.3 | 3034.7 |
| SpinalCord | Dmax | 4143.7 | 3808.5 | 3872.8 |
| SpinalCord + 5 | Dmax | 5149.3 | 5199.8 | 4987.7 |
| mm | | | | |
| Submandibular_{—} | Dmean | 6646.2 | 6694.4 | 6805 |
| L | | | | |
| Submandibular_{—} | Dmean | 6294.4 | 6345.8 | 6446 |
| R | | | | |
| TemporalLobe_L | Dmean | 420.5 | 100.9 | 284.4 |
| | D1cc | 5028.4 | 3965 | 5109.6 |
| TemporalLobe_R | Dmean | 507.4 | 237.4 | 469.1 |
| | D1cc | 5647.6 | 5881.6 | 5980.8 |

| | | | |
|---|---|---|---|
| CI | 1.41 | 1.34 | 1.39 |

### Dose Rate and Delivery Efficiency

Another advantage of RAPIDSPARC™ is its fast delivery speed. We evaluated the dose rate coverage for different arc therapy methods using a concept called Dose Threshold Dose Rate (DTDR) (Kang et al. 2021). As shown in Equation (11), for each voxel (a small volume element within the target area), the DTDR is defined as the minimum instantaneous dose rate delivered by any beam contributing a dose above a predefined threshold d* to that voxel. \dot{D}ⱼ represents the DTDR for voxel j, \dot{D}ᵢⱼ represents the dose rate delivered to voxel j by the i-th beam, d* is the predefined dose threshold as 5% of the central axial of a spot.

$${\dot{D}}_{J} = min ( {\dot{D}}_{ij} ) , if {D}_{ij} > {d}^{*} , i = 1 , 2 … n , \qquad ( eq . 11 )$$

Where the \dot{D}ᵢⱼ is,

$${\dot{D}}_{ij} = {\dot{D}}_{max} {e}^{- \frac{{( {r}_{j} - {r}_{i}^{c} )}^{2}}{{σ}^{2}}} , \qquad ( eq . 12 )$$

Dₘₐₓ is the max dose rate at the spot center, rⱼ represents the position of the j-th voxel and rᵢ^{c} is the position of the i-th spot center, σ is the spot size of the i-th spot.

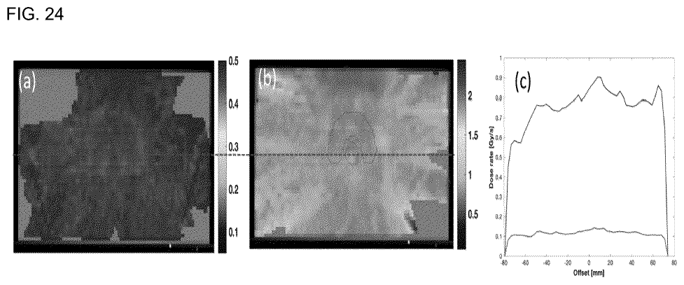

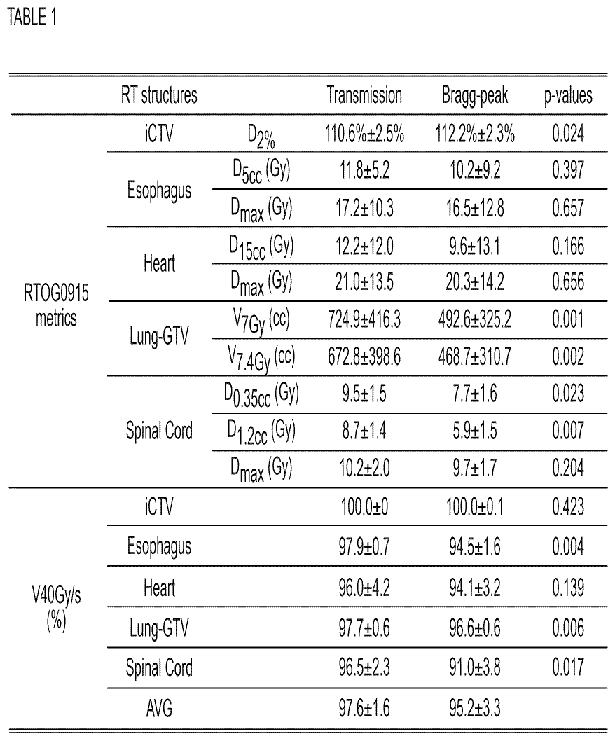

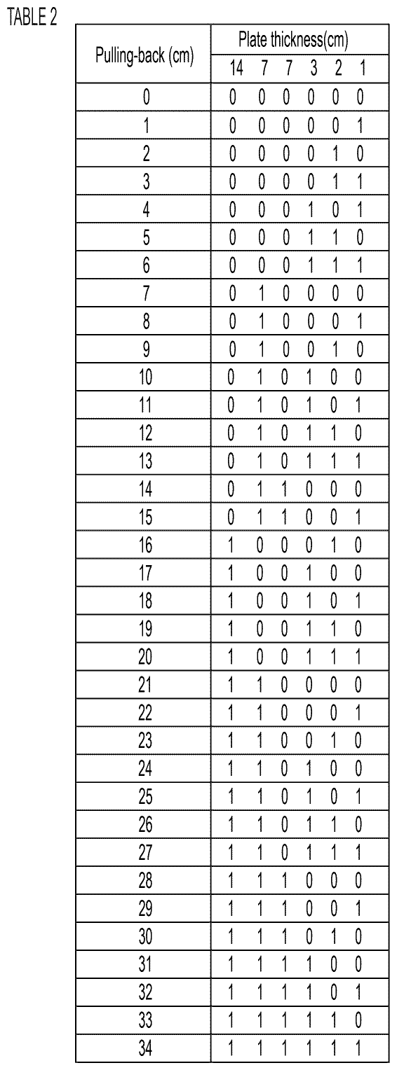

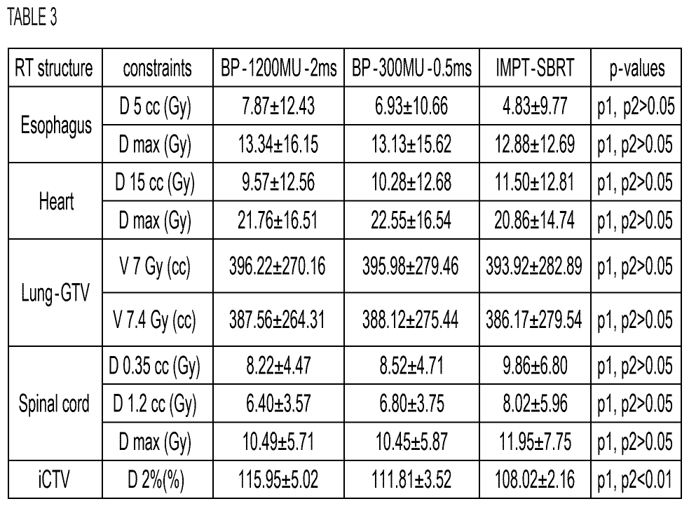

The conventional ARC used a MMU of 3, compared to the RAPIDSPARC™ used a MMU 60, the 2D dose rate distribution of the RAPIDSPARC™ is much higher compared to the ARC. As shown in FIG. 24(c), the RAPIDSPARC™ can delivery as fast as 8 times than the ARC method using conventional MMU and beam current operational parameters.

### The Robustness Analysis in Patient Planning

The robustness analysis used 3.5% range uncertainties and 3 mm setup uncertainties. Due to the wide Bragg peak, with D80-80 of the Bragg peak being about 10 mm, RAPIDSPARC™ is less sensitive to range uncertainties and day-to-day setup discrepancies. This results in a more robust plan quality.

### Discussion

Replacing the energy selection system with URS introduces additional materials into the beamline, particularly in the nozzle, which results in an increased spot size. One approach to mitigate this is to use a lower beam energy, which would reduce the range shifter thickness and scattering dose. Adding an aperture or multi-leaf collimator (MLC) downstream of the range shifter can further sharpen the penumbra.

The study only tested the algorithm on a phantom and on prostate and head and neck treatment sites. More research is needed to determine the technique's effectiveness across a wider variety of tumors. The study suggests that incorporating a fixed aperture or MLC into the beamline could reduce the dose penumbra. Future research could investigate the integration of an MLC into the system and its impact on treatment planning and delivery.

### Conclusion

This study investigated the feasibility of using quasi-distal tracking with a single-energy layer for proton rapid spot arc therapy. Our findings demonstrate that this technique can achieve dosimetry equivalent to conventional mono-energy arc plans while offering significantly higher delivery efficiency. This translates to much faster treatment, potentially improving patient comfort and reducing machine occupancy.

Both the phantom and several clinical cases demonstrated the potential for clinical application. By simplifying the treatment delivery process and enhancing efficiency, this approach paves the way for broader clinical adoption of proton arc therapy.

Further research is warranted to validate these findings across a wider range of clinical scenarios. Additionally, investigations into integrating this technique with existing machines and conducting thorough measurements are crucial for successful clinical implementation. Overall, this study highlights the promise of quasi-distal tracking with a single-energy layer in making proton arc therapy a more efficient and accessible treatment option for cancer patients.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and it is not intended to limit the scope of the present disclosure, which is defined solely by the claims. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

### Disclosed Embodiments:

Embodiment 1. A system for delivering radiation therapy, comprising:
   an ionizing radiation source configured to produce single-energy beams to form at least two fields of shifted and compensated ionizing radiation;
   a treatment head configured to deliver the single-energy beams to the target tissue, wherein the treatment head comprises:
   at least one universal range shifter adjusted to shift a range of the single-energy beams so that Bragg peak of the single-energy beams coincides with the target tissue, and
   at least one range compensator adjusted to compensate the range of the single-energy beams so that the Bragg peak of the single-energy beams coincide with a contour of the target tissue.
Embodiment 2. The system of embodiment 1, wherein said system does not comprise an energy selection system.
Embodiment 3. The system of embodiment 1, further comprising a fixed non-rotatable gantry with a rotatable chair.
Embodiment 4. The system of embodiment 1, wherein the single-energy beams comprise at least one of protons, helium, carbon, argon, or neon.
Embodiment 5. The system of embodiment 1, wherein the single-energy beams comprise protons.
Embodiment 6. The system of embodiment 1, wherein the single-energy beams comprise the highest energy beams directly from the ionizing radiation source.
Embodiment 7. The system of embodiment 1, wherein
   the at least one universal range shifter comprises a solid water-based range shifter,
   the at least one range compensator is generated using 3D printing technology, and
   centers of the at least one range compensators are aligned to radiation centers of the single-energy beams.
Embodiment 8. A method for delivering radiation therapy, comprising:
   producing, by an ionizing radiation source, single-energy beams to form at least two fields of shifted and compensated radiation, wherein the ionizing radiation source is directly integrated into a treatment gantry;
   guiding, by at least one bending magnets, the single-energy beams to a target tissue; and delivering, by a treatment head, the single-energy beams to the targe tissue by:
   shifting, by at least one universal range shifter, a range of the single-energy beams so that Bragg peak of the single-energy beams coincides with the target tissue, and
   compensating, by at least one range compensators, the range of the single-energy beams so that the Bragg peak of the single-energy beams coincides with a contour of the target tissue.
Embodiment 9. The method of embodiment 8, wherein said method does not comprise reducing energy of the single-energy beams by an energy degradation system.
Embodiment 10. The method of embodiment 8, wherein the treatment gantry is a fixed non-rotatable gantry with a rotatable chair.
Embodiment 11. The method of embodiment 8, wherein the single-energy beams comprise at least one of protons, helium, carbon, argon, or neon.
Embodiment 12. The method of embodiment 11, wherein the single-energy beams comprise protons.
Embodiment 13. The method of embodiment 8, wherein the single-energy beams are produced directly from the ionizing radiation source.
Embodiment 14. The method of embodiment 8, wherein
   the at least one range compensators are generated using 3D printing technology, and
   centers of the at least one range compensators are aligned to radiation centers of the single-energy beams.
Embodiment 15. The method of embodiment 8, wherein an inverse-planning optimization protocol is used to determine number, position and thickness of the at least one universal range shifter.
Embodiment 16. The method of embodiment 15, wherein the inverse-planning optimization protocol is used to further determine shapes of the at least one range compensator to match the contour of the target tissue.
Embodiment 17. The method of embodiment 16, wherein the inverse-planning optimization protocol is used to further determine a dose rate characterized using a hybrid method.
Embodiment 18. The method of embodiment 17, wherein the hybrid method combines a measured time structure of delivered spots with a measured dose to reconstruct a three-dimensional dose rate distribution.
Embodiment 19. The method of embodiment 18, wherein the three-dimensional dose rate distribution is verified using convolution superposition (PCS) and/or Monte Carlo (MC) algorithms.
Embodiment 20. A system for delivering radiation therapy comprising:
   an ionizing radiation source configured to produce single-energy beams; and
   a treatment head comprising at least one universal range shifter and at least one range compensator;
   wherein the high intensity of the beams can be preserved between the ionizing radiation source and the treatment head.
Embodiment 21. The system of embodiment 20, wherein said single-energy beams comprise the maximum energy beams from the ionizing radiation source.
Embodiment 22. The system of embodiment 21, wherein said ionizing radiation source comprises a cyclotron or synchrotron or synchrocyclotron or other type accelerators.
Embodiment 23. A method for delivering radiation therapy comprising use of the system of embodiment 22 to administer said single-energy beams to a target tissue.
Embodiment 24. The method of embodiment 23, wherein an inverse-planning optimization protocol is used to determine the number, position and thickness of the at least one universal range shifter.
Embodiment 25. The method of embodiment 24, wherein the inverse-planning optimization protocol is used to further determine the shape of the at least one range compensator to match the contour of the target tissue.
Embodiment 26. The method of embodiment 24, wherein the inverse-planning optimization protocol is used to further determine a dose rate characterized using a hybrid method.
Embodiment 27. A system for delivering radiation therapy comprising:
   an ionizing radiation source; and
   a treatment head comprising at least one universal range shifter and at least one range compensator;
   wherein the the system does not comprise an energy selection system between the ionizing radiation source and the treatment head.
Embodiment 28. A system for delivering radiation arc therapy to a target tissue, comprising:
   an ionizing radiation source configured to produce single-energy beams to form at least two fields of shifted and compensated ionizing radiation;
   a treatment head configured to deliver the single-energy beams to the target tissue, wherein the treatment head comprises:
   at least one universal range shifter adjusted to shift a range of the single-energy beams so that Bragg peak of the single-energy beams coincides with the target tissue.
Embodiment 29. The system of embodiment 28, wherein said system does not comprise an energy selection system.
Embodiment 30. The system of embodiment 28, further comprising a fixed non-rotatable gantry with a rotatable chair.
Embodiment 31. The system of embodiment 28, wherein the single-energy beams comprise at least one of protons, helium, carbon, argon, or neon.
Embodiment 32. The system of embodiment 28, wherein the single-energy beams comprise protons.
Embodiment 33. The system of embodiment 28, wherein the single-energy beams comprise the highest energy beams directly from the ionizing radiation source.
Embodiment 34. A method for delivering radiation arc therapy to a target tissue, comprising:
   providing an ionizing radiation source configured to produce single-energy beams to form at least two fields of shifted and compensated ionizing radiation;
   wherein said ionizing radiation passes through a treatment head configured to deliver the single-energy beams to the target tissue, wherein the treatment head comprises:
   at least one universal range shifter adjusted to shift a range of the single-energy beams so that Bragg peak of the single-energy beams coincides with the target tissue.
Embodiment 35. The method of embodiment 34, wherein said system does not comprise an energy selection system.
Embodiment 36. The method of embodiment 34, further comprising a fixed non-rotatable gantry with a rotatable chair.
Embodiment 37. The method of embodiment 34, wherein the single-energy beams comprise at least one of protons, helium, carbon, argon, or neon.
Embodiment 38. The method of embodiment 34, wherein the single-energy beams comprise protons.
Embodiment 39. The method of embodiment 34, wherein the single-energy beams comprise the highest energy beams directly from the ionizing radiation source.

1. A system for delivering radiation therapy, comprising:
an ionizing radiation source configured to produce single-energy beams to form at least two fields of shifted and compensated ionizing radiation;
a treatment head configured to deliver the single-energy beams to the target tissue, wherein the treatment head comprises:
at least one universal range shifter adjusted to shift a range of the single-energy beams so that Bragg peak of the single-energy beams coincides with the target tissue, and
at least one range compensator adjusted to compensate the range of the single-energy beams so that the Bragg peak of the single-energy beams coincide with a contour of the target tissue.

2. The system of claim 1, wherein said system does not comprise an energy selection system.

3. The system of claim 1, further comprising a fixed non-rotatable gantry with a rotatable chair.

4. The system of claim 1, wherein the single-energy beams comprise at least one of protons, helium, carbon, argon, or neon.

5. The system of claim 1, wherein the single-energy beams comprise protons.

6. The system of claim 1, wherein the single-energy beams comprise the highest energy beams directly from the ionizing radiation source.

7. The system of claim 1, wherein the at least one universal range shifter comprises a solid, water-based range shifter,
the at least one range compensator is generated using 3D printing technology, and centers of the at least one range compensators are aligned to radiation center of the single-energy beams.

8. A method for delivering radiation therapy, comprising:
producing, by an ionizing radiation source, single-energy beams to form at least two fields of shifted and compensated radiation, wherein the ionizing radiation source is directly integrated into a treatment gantry;
guiding, by at least one bending magnet, the single-energy beams to a target tissue; and
delivering, by a treatment head, the single-energy beams to the targe tissue by:
shifting, by at least one universal range shifter, a range of the single-energy beams so that Bragg peak of the single-energy beams coincides with the target tissue, and
compensating, by at least one range compensator, the range of the single-energy beams so that the Bragg peak of the single-energy beams coincides with a contour of the target tissue.

9. The method of claim 8, wherein said method does not comprise reducing energy of the single-energy beams by an energy degradation system.

10.-14. (canceled)

15. The method of claim 8, wherein an inverse-planning optimization protocol is used to determine number, position and thickness of the at least one universal range shifter.

16. The method of claim 15, wherein the inverse-planning optimization protocol is used to further determine shapes of the at least one range compensators to match the contour of the target tissue.

17. The method of claim 16, wherein the inverse-planning optimization protocol is used to further determine a dose rate characterized using a hybrid method.

18. The method of claim 17, wherein the hybrid method combines a measured time structure of delivered spots with a measured dose to reconstruct a three-dimensional dose rate distribution.

19. The method of claim 18, wherein the three-dimensional dose rate distribution is verified using convolution superposition (PCS) and/or Monte Carlo (MC) algorithms.

20.-27. (canceled)

28. A system for delivering radiation arc therapy to a target tissue, comprising:
an ionizing radiation source configured to produce single-energy beams to form at least two fields of shifted and compensated ionizing radiation;
a treatment head configured to deliver the single-energy beams to the target tissue, wherein the treatment head comprises:
at least one universal range shifter adjusted to shift a range of the single-energy beams so that Bragg peak of the single-energy beams coincides with the target tissue.

29. The system of claim 28, wherein said system does not comprise an energy selection system.

30. The system of claim 28, further comprising a fixed non-rotatable gantry with a rotatable chair.

31. The system of claim 28, wherein the single-energy beams comprise at least one of protons, helium, carbon, argon, or neon.

32. The system of claim 28, wherein the single-energy beams comprise protons.

33. The system of claim 28, wherein the single-energy beams comprise the highest energy beams directly from the ionizing radiation source.

34.-39. (canceled)
